(19) United States
(12) Patent Application Publication
(10) Pub. No.: US 2020/0289321 A1
(43) Pub. Date: Sep. 17, 2020

(54) Circadian Rhythm Adjustment System

(71) Applicant: LumosTech, Inc., San Francisco, CA (US)

(72) Inventor: Biquan Luo, San Francisco, CA (US)

(73) Assignee: LumosTech, Inc., San Francisco, CA (US)

(21) Appl. No.: 16/810,800

(22) Filed: Mar. 5, 2020

Related U.S. Application Data
(63) Continuation-in-part of application No. 16/803,961, filed on Feb. 27, 2020.
(60) Provisional application No. 62/812,683, filed on Mar. 1, 2019.
(60) Provisional application No. 62/814,257, filed on Mar. 5, 2019.
(60) Provisional application No. 62/835,473, filed on Apr. 17, 2019.
(60) Provisional application No. 62/814,257, filed on Mar. 5, 2019.
(60) Provisional application No. 62/835,473, filed on Apr. 17, 2019.

(51) Int. Cl. A61F 9/04; A61N 5/06; A61M 21/02

(52) U.S. Cl. CPC A61F 9/04; A61N 5/0618; A61M 2021/0044; A61N 2005/063; A61N 2005/0648; A61M 21/02

(57) ABSTRACT

A system is disclosed in which an application runs may on a mobile device (or other computing device), which presents an interface to a user for entering information related to circadian rhythm. Based on the circadian information received a circadian clock adjustment model is computed, which is used for determining the lighting pattern presented to a user for treating circadian rhythm disorder or adjusting a user's circadian rhythm. The lighting pattern may be produced in the form of light flashes via lights that may be mounted in a sleep mask, glasses frame, other wearable device, mobile device, display, or lighting system of a room. Information related to the circadian rhythm may be detected (e.g., via sensors) and update the circadian rhythm model based in the information detected.

### RELATED APPLICATIONS

This Application is a continuation-in-part of U.S. patent application Ser. No. 16/803961, filed Feb. 27, 2020, by Biquan Luo, which in turn claims priority benefit to claims priority benefit to

U.S. provisional Application Ser. No. 62/812,683 (Docket # D6-2), entitled “ADJUSTABLE MASK,” filed Mar. 1, 2019, by Biquan Luo;

U.S. provisional Application Ser. No. 62/814,257 (Docket # D6-1), entitled “Circadian Rhythm Adjustment System,” filed Mar. 5, 2019, by Biquan Luo; and

U.S. provisional Application Ser. No. 62/835,473 (Docket # D6-3), entitled “Circadian Rhythm Adjustment System,” filed Apr. 17, 2019, by Biquan Luo;

this Application also claims priority benefit to

U.S. provisional Application Ser. No. 62/814,257 (Docket # D6-1), entitled “Circadian Rhythm Adjustment System,” filed Mar. 5, 2019, by Biquan Luo; and

U.S. provisional Application Ser. No. 62/835,473 (Docket # D6-3), entitled “Circadian Rhythm Adjustment System,” filed Apr. 17, 2019, by Biquan Luo.

All of the above Applications are incorporated herein by reference.

### FIELD OF THE INVENTION

This specification relates to the field of methods and devices for treating circadian rhythm disorders.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Circadian rhythms are physiological and behavioral oscillations that are normally synchronized with the natural light-dark cycle of the day. Circadian rhythm disorders happen when circadian rhythms are out of synchronization with the actual sleep-wake schedules. Circadian rhythm disorders typically are related to sudden and/or extreme changes in the relationship between an organism's exposure to environmental light and the organism's activity. For example, circadian rhythm disorders are known to be associated with changes in geographical location (jet lag) and night activity (graveyard shift workers). Another common type of circadian rhythm disorder is seasonal affective disorder (SAD), which is characterized by symptoms such as depression during the winter seasons, when the duration of daylight is reduced. Circadian rhythms in humans and other mammals are affected by the exposure of the retina to light. Various techniques and devices have been developed to treat circadian rhythm disorders by exposing the eyes to light.

### BRIEF DESCRIPTION

In the following drawings like reference numbers are used to refer to like elements. Although the following figures depict various examples of the invention, the invention is not limited to the examples depicted in the figures.

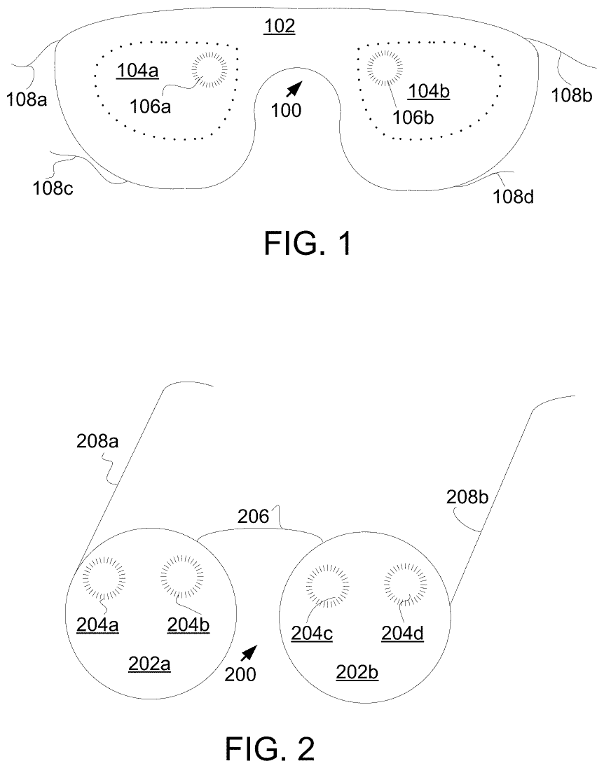

FIG. 1 shows an embodiment of a mask having circadian rhythm treatment hardware.

FIG. 2 shows an embodiment of glasses in which the circadian rhythm treatment hardware is built into the frames and/or glasses.

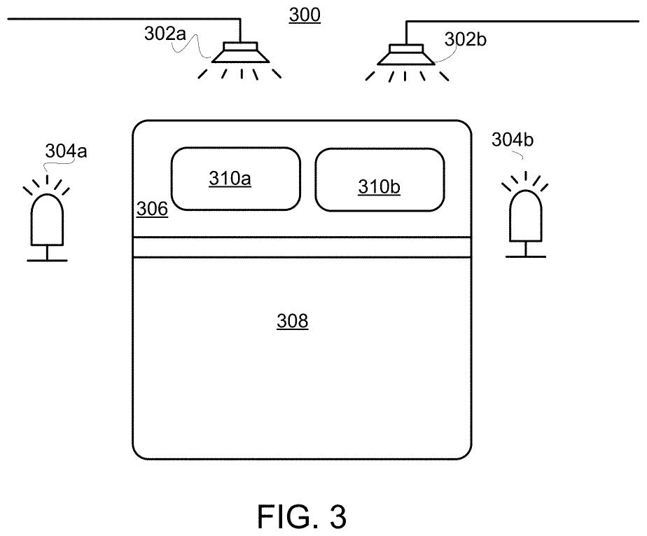

FIG. 3 shows an embodiment in which the circadian rhythm treatment hardware is built into a lighting system of the room of a building, for example.

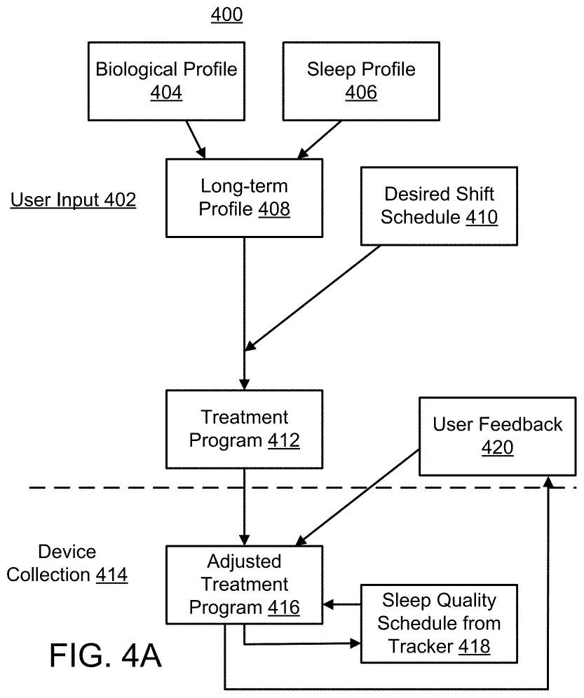

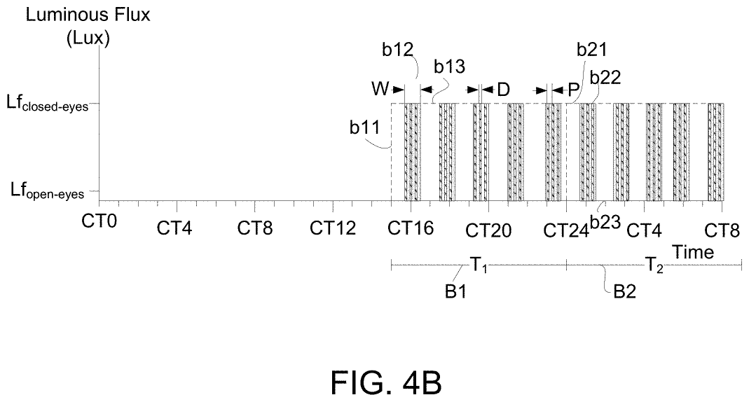

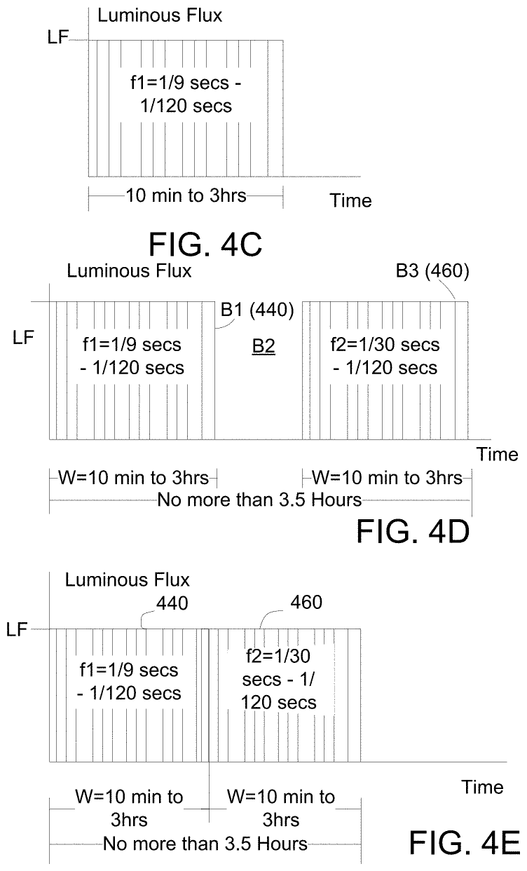

FIG. 4A shows a flowchart for a method in which the system of FIG. 4A may operate.

FIG. 4B shows an example of a treatment regimen for treating circadian rhythm, which may include treatment periods T1 and T2.

FIGS. 4C-4E shows an example of one or more blocks of light flashes having a block width W within a treatment window.

FIG. 4C shows an example of a block of light flashes that have a frequency of f1= 1/9 second to 1/120 seconds.

FIGS. 4D and E show examples of two consecutive series of flashes, in which each block of flashes is of a different frequency.

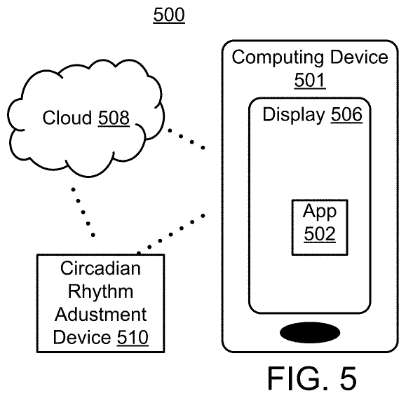

FIG. 5 shows a block diagram of an embodiment of the circadian rhythm therapy system.

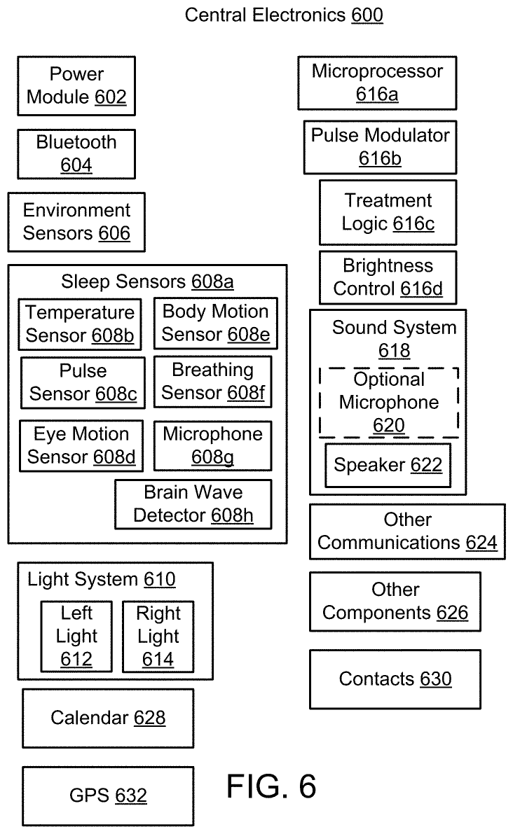

FIG. 6 shows a block diagram of a system for adjusting a circadian rhythm.

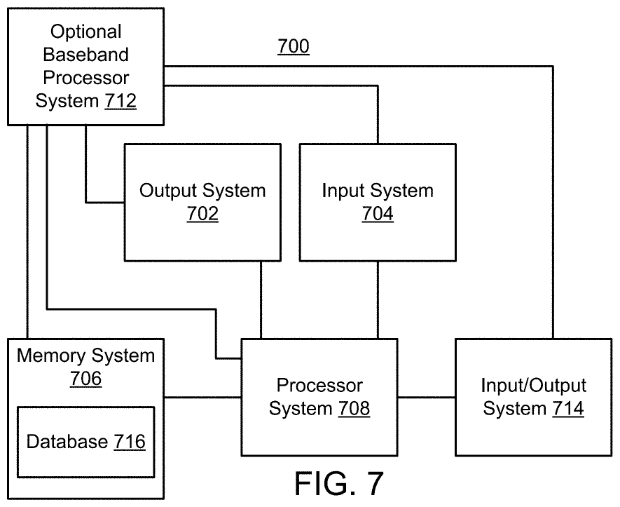

FIG. 7 shows a block diagram of a mobile device/computing device used in the system of FIG. 1.

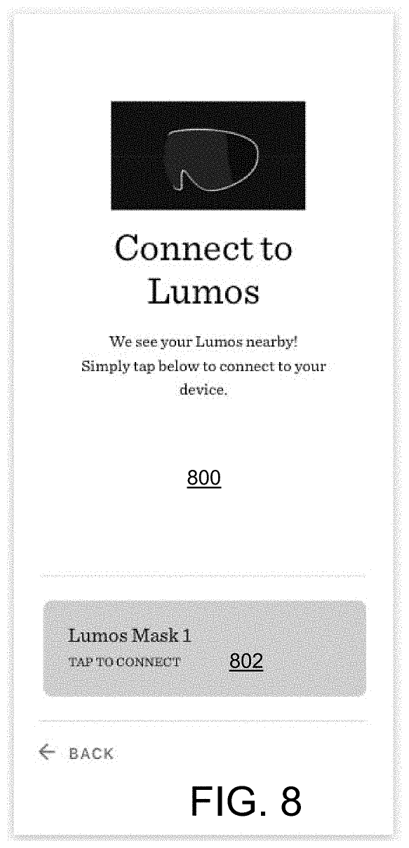

FIG. 8 shows an example of a screenshot of an embodiment of page for communicatively connecting the system of FIGS. 1, 2, and/or 3 to the system of FIG. 5, prior to the communication being established.

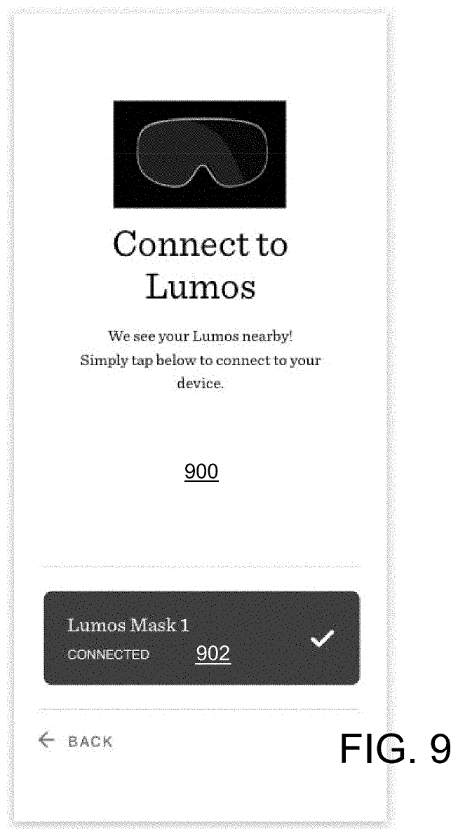

FIG. 9 shows an example of a screenshot of an embodiment of page for communicatively connecting the system of FIGS. 1, 2, and/or 3 to the system of FIG. 5, after the communication was established.

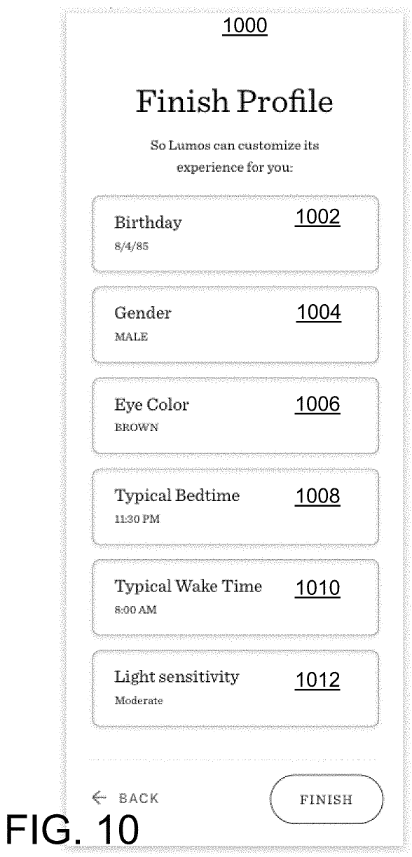

FIG. 10 shows an example of a page from the mobile application for entering personal information for personalizing the application.

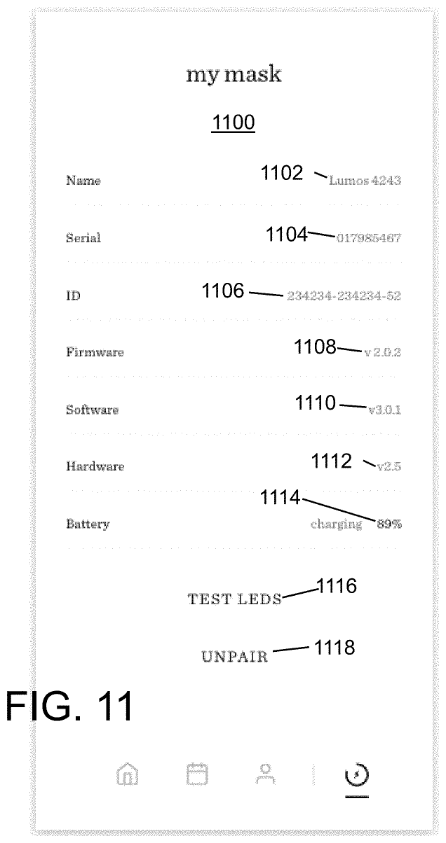

FIG. 11 shows an example of a page from the mobile application for entering and/or displaying information for identifying the hardware and software of the system of FIG. 1, 2 or 3.

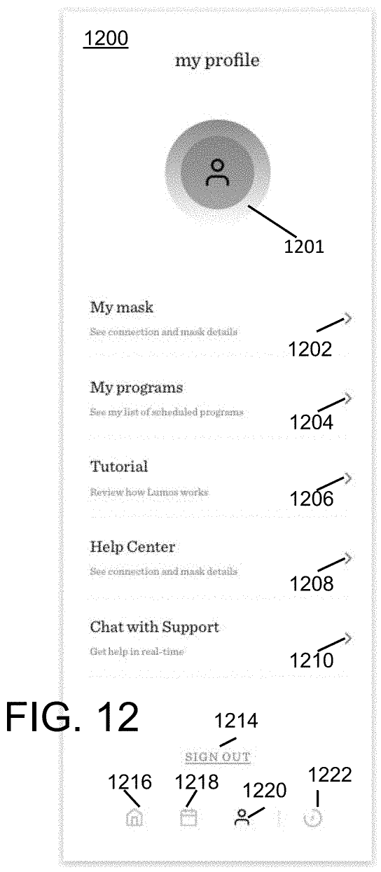

FIG. 12 shows an example of a page from the mobile application for viewing or editing the profile information of FIGS. 10 and/or 11 or viewing or editing information related to a sleep schedule and/or the use of the system.

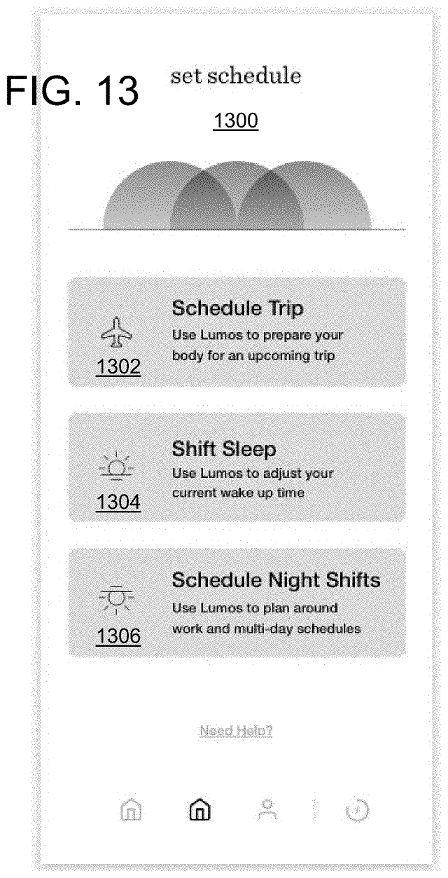

FIG. 13 is an example of schedule page from the mobile application.

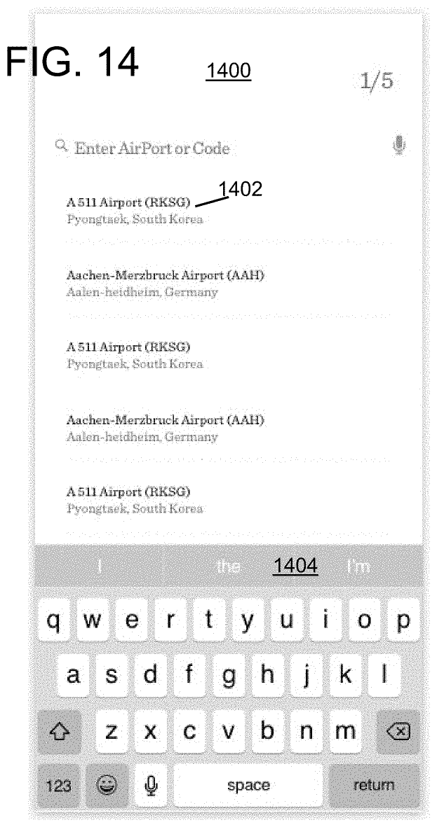

FIG. 14 shows an example of a page from the mobile application for choosing the locations that the user plans to visit.

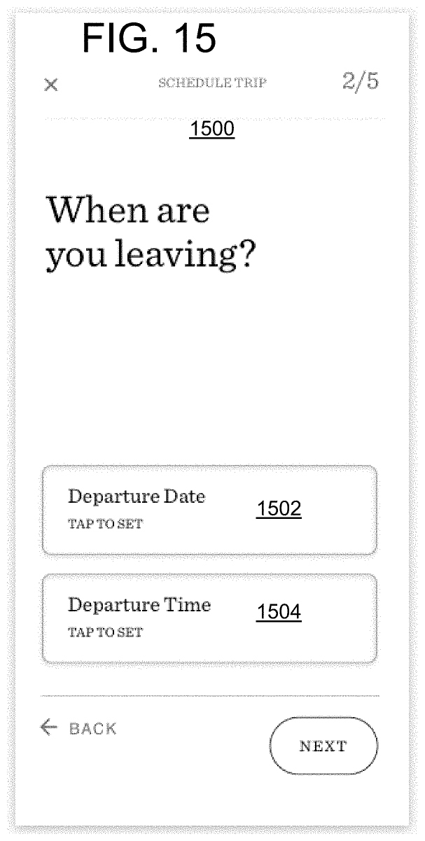

FIG. 15 shows an example of a page from the mobile application for choosing departure date and time for a particular location that the user intends to visit.

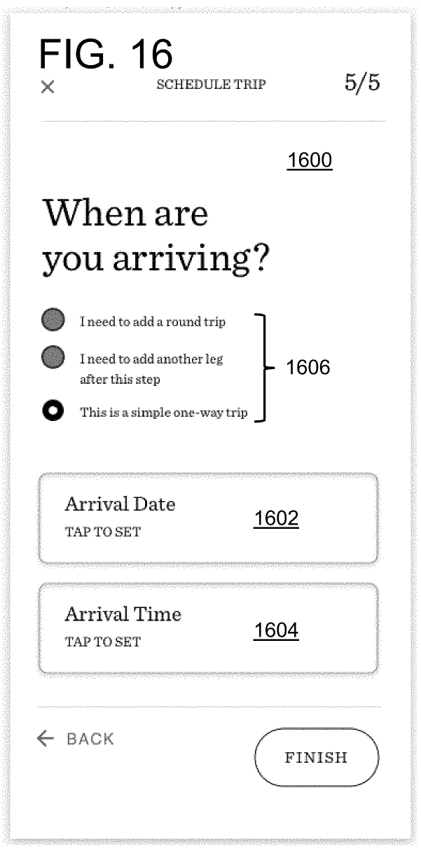

FIG. 16 shows an example of a page from the mobile application for choosing an arrival date and time for a particular location that the user intends to visit.

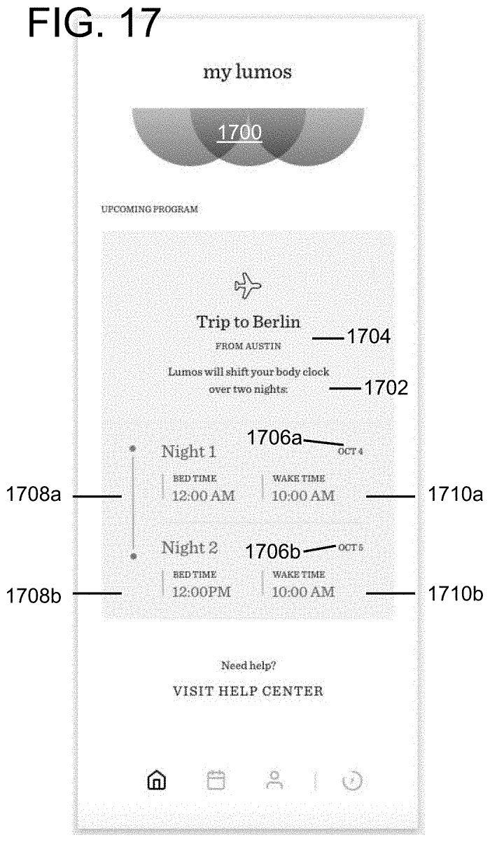

FIG. 17 shows an example of a page from the mobile application showing a circadian rhythm adjusting program for reducing jet lag after the trip has been entered.

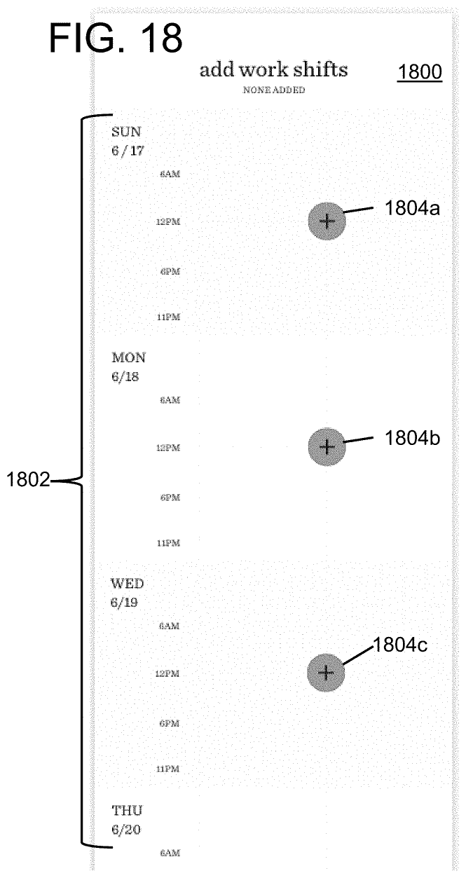

FIG. 18 shows an example of a portion of a page from the mobile application showing a calendar for entering work shift schedules that occur at various times, prior to scheduling the shifts.

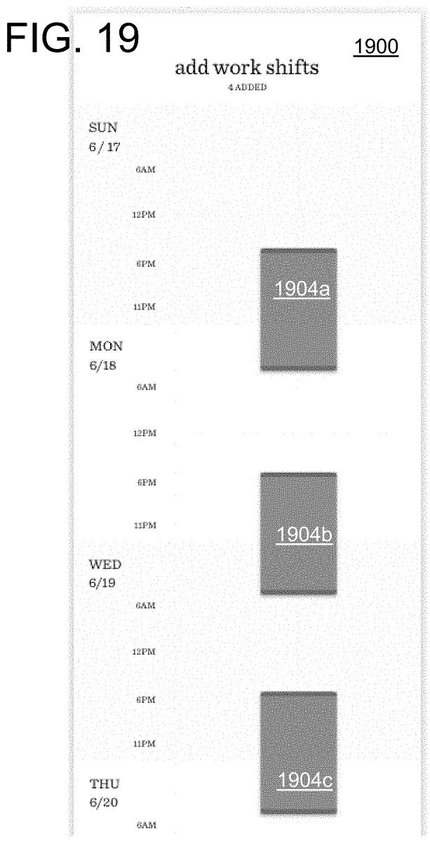

FIG. 19 shows an example of a portion of a page from the mobile application showing calendar for entering work shift schedule that occur at various times, after some shifts have been scheduled.

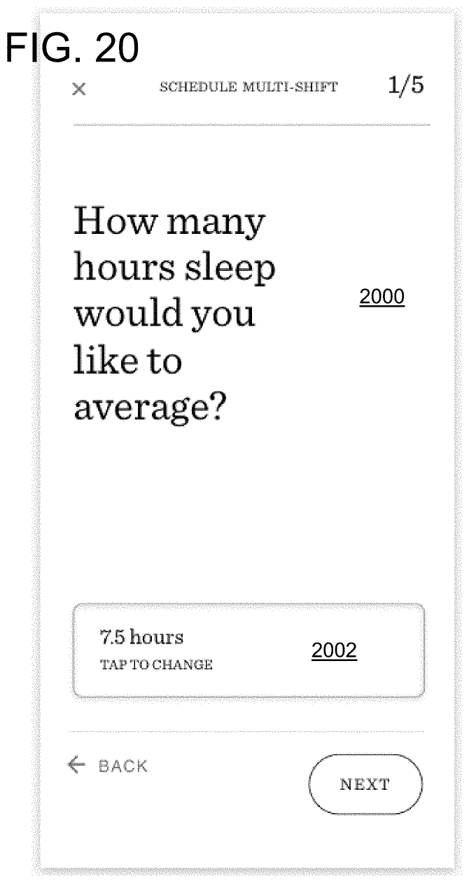

FIG. 20 shows an example of a page from the mobile application having a field for entering a desired number of hours the user would like to sleep between the shifts.

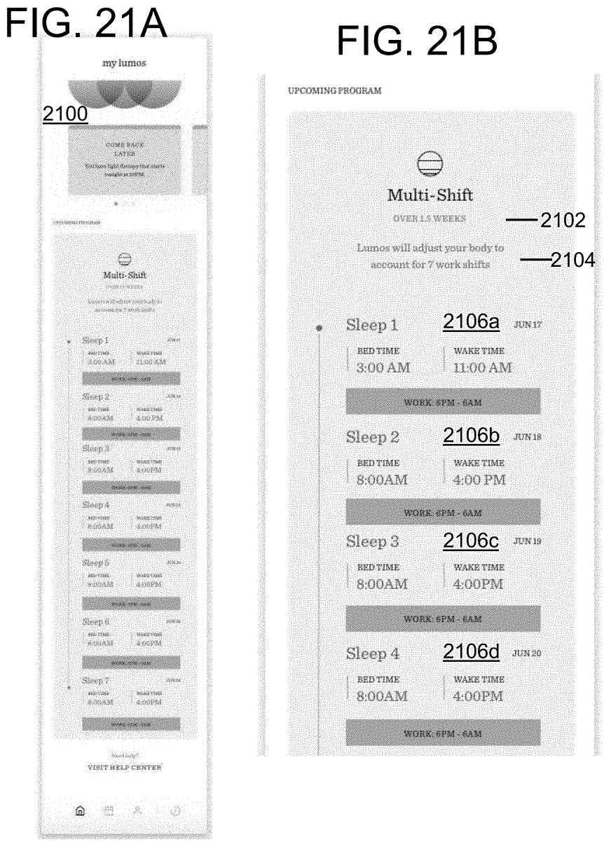

FIG. 21A shows an example of a page from the mobile application showing a circadian rhythm adjusting program for the night shifts after the schedule has been entered. FIG. 21B shows a portion of the page in FIG. 21A.

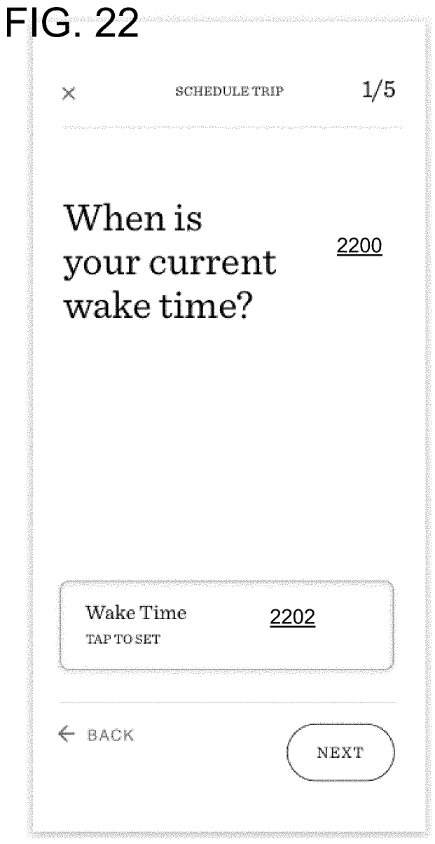

FIG. 22 shows an example of a page from the mobile application for the user's current wake time if they want to change their sleep schedules.

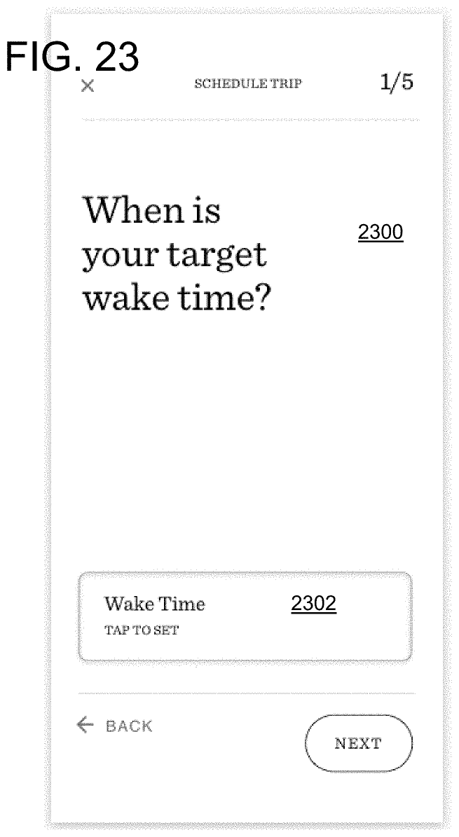

FIG. 23 shows an example of a page from the mobile application for the user's target wake time if they want to change their sleep schedules.

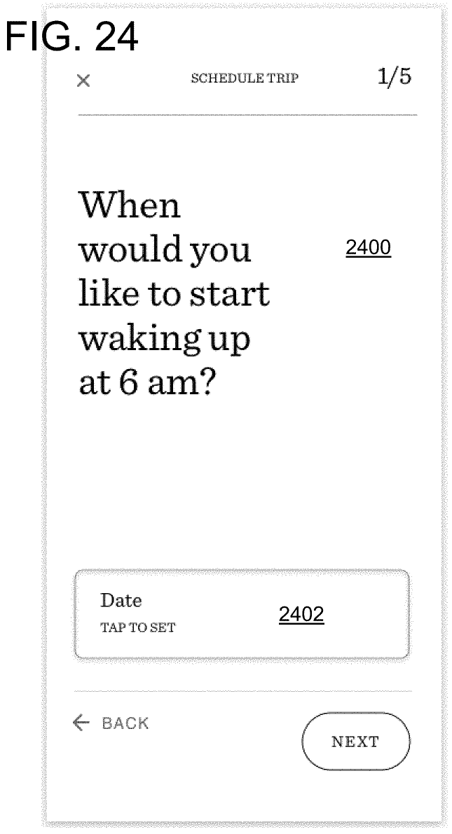

FIG. 24 shows an example of a page from the mobile application for choosing when the user wants to start changing their sleep schedules.

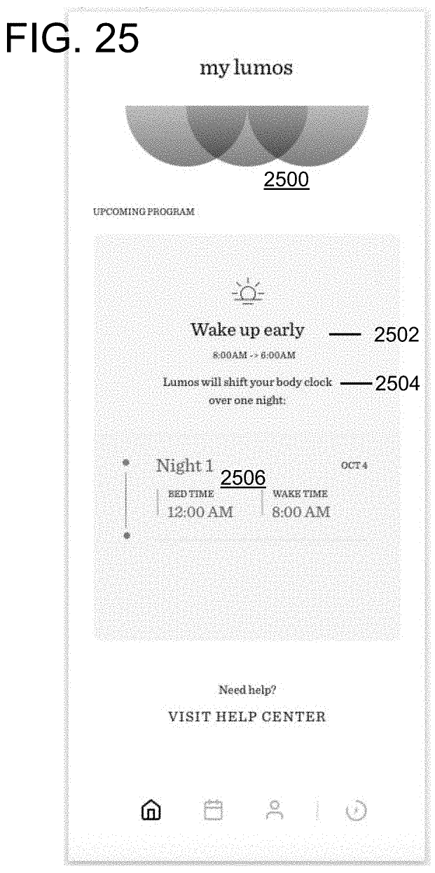

FIG. 25 shows an example of a page from the mobile application showing a circadian rhythm adjusting program for changing sleep schedules after the schedule has been entered.

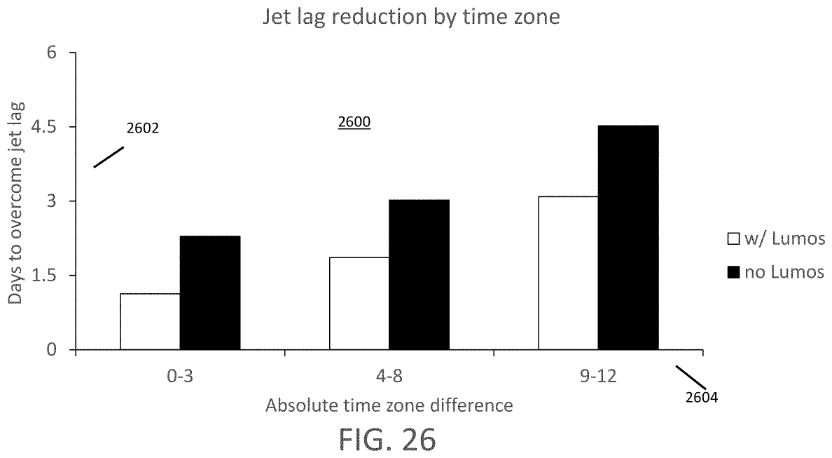

FIG. 26 shows a bar chart comparing the number of days to get over from jet lag based on the number of time zones traversed, with and without the system of FIGS. 1-11.

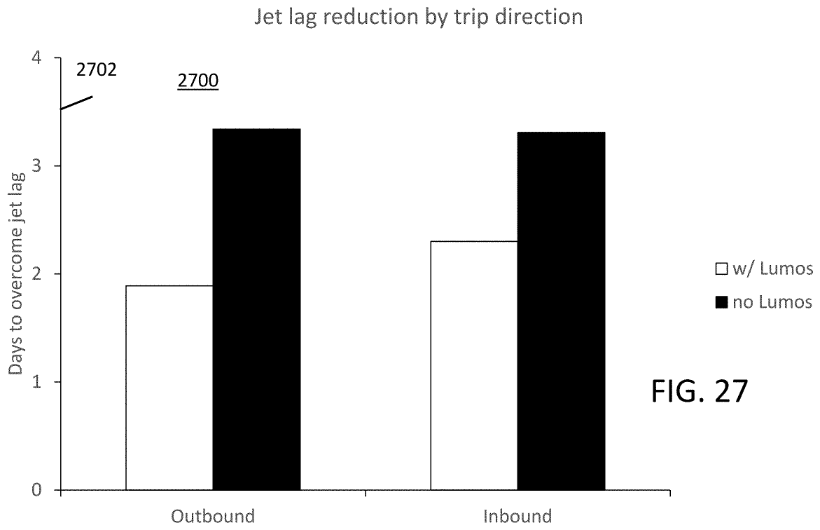

FIG. 27 shows a bar chart comparing the number of days to recover from jet lag, based the direction of the trip, with and without an embodiment of the system of FIGS. 1-11.

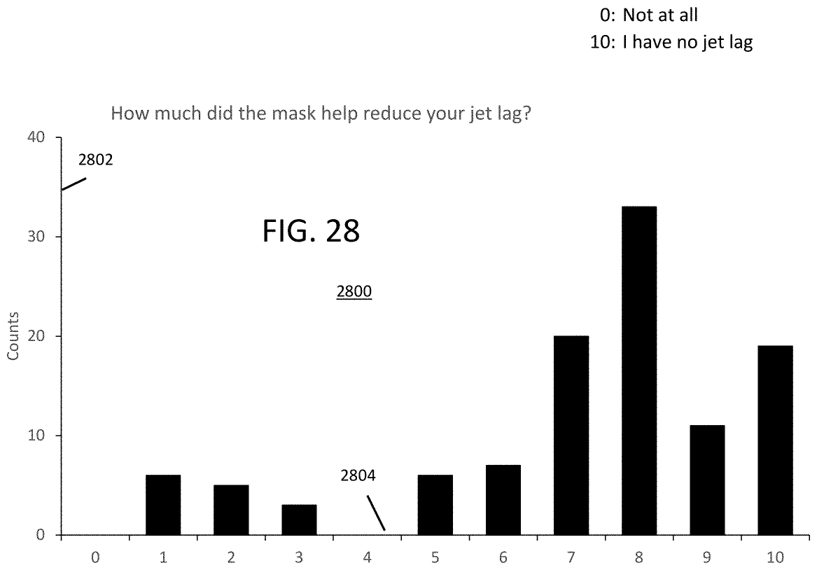

FIG. 28 shows a first chart of how much users rated an embodiment of the system of FIGS. 1-11 in reducing jet lag.

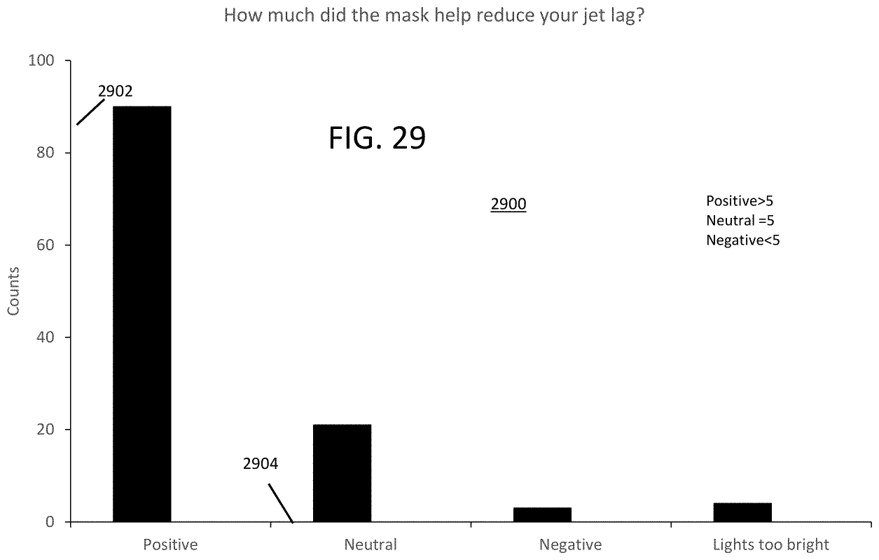

FIG. 29 shows a second chart, based on the data of used for FIG. 23, of how users rated an embodiment of the system of FIGS. 1-11 in reducing jet lag.

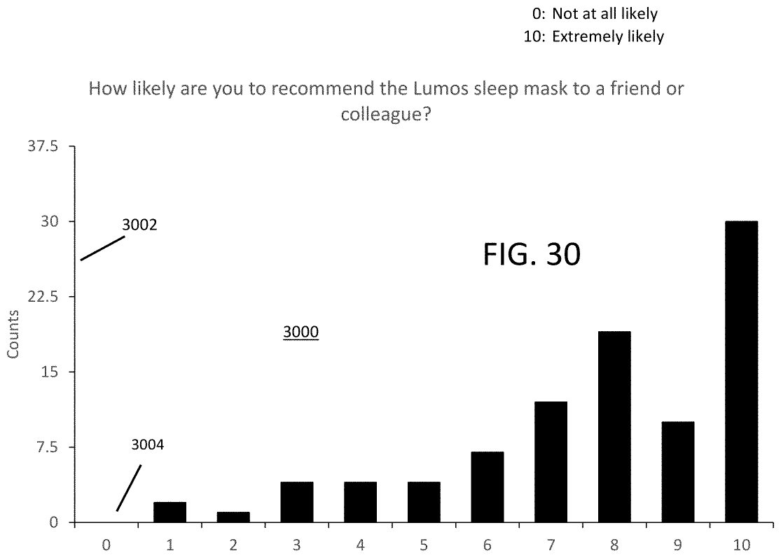

FIG. 30 shows a bar chart of whether users would recommend an embodiment of the system of FIGS. 1-11.

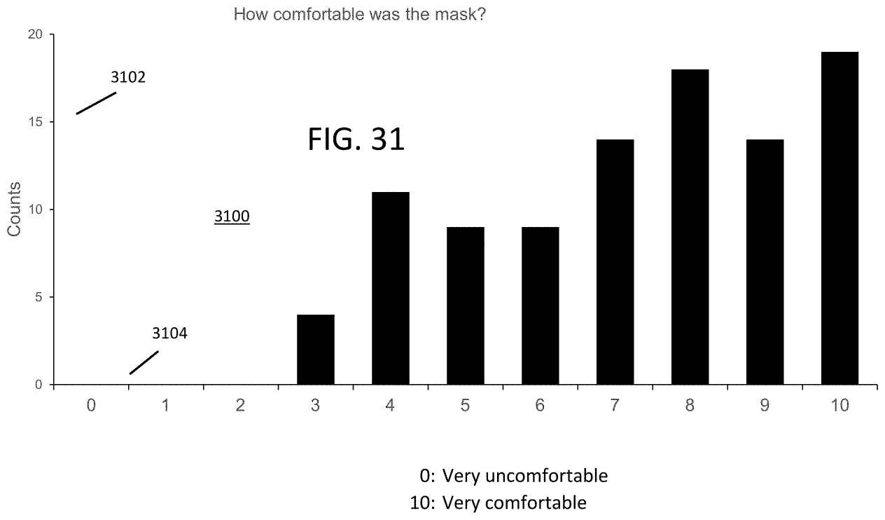

FIG. 31 shows how users rated an embodiment of the system of FIGS. 1-11 for comfort.

### DETAILED DESCRIPTION

Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

The disclosures of the devices and methods of this application and the appendix describe different parts of what can be the same devices, methods, and/or systems. Any element of this may be used in the embodiments of the appendix and any element of appendix may be used in the embodiments of this application.

FIG. 1 shows an embodiment system 100. System 100 may include mask 102, cupped regions 104a and 104b, lights 106a and 106b, and straps 108a, 108b, 108c, and 108d.

System 100 may be a mask having circadian rhythm treatment hardware (e.g., LEDs, electronics, and sensors) for computing and adjusting a ciradian rhythm treatment.

### 1. System

This specification relates to systems and methods for treating and preventing circadian rhythm disorders, which may present itself as jet lag, disorders related to shift works, seasonal affective disorder (SAD), and related conditions.

A system to treat/prevent circadian misalignment by actively shifting circadian phases during sleep is presented. In alternative embodiments, the circadian rhythm may be adjusted while the user is not sleeping in addition to or instead of adjusting the circadian rhythm during sleep. The phase shift may be caused by applying light pulses, which may be generated based on a treatment regimen, which may be controlled by a software program, during sleep. The system may include software, hardware, a light source, such as an LED, and/or user interface, for example. The system may include a light calculation module, which implements light calculation logic, which determines a pattern of light to apply to the user. The hardware is discussed further, below.

Form factors may include sleep masks, and/or different sizes and shapes of other things worn (or that may accompany of a person while sleeping). The system may be integrated into a variety of devices of systems of a variety of sizes and shapes.

Currently, inventions on the market use continuous light stimulation applied while the users are awake in order to shift circadian phases. In contrast to other approaches, in an embodiment, the user may use light flashes while the user is sleeping, to adjust the user's circadian rhythm. As a result of using light pulses, and as a result of light pulses being less invasive and less disruptive to sleep, the system may potentially shift circadian rhythm while the users are sleeping. Additionally, compared to other light therapy devices that may shift the circadian rhythm, the use of light pulses is less disruptive to users' day-time activities, also, and therefore potentially be more convenient. Due to retinal physiology and the sensitivity of the circadian adjustment system in the brain, light flash stimulation at night can potentially be more effective in shifting circadian rhythm than continuous light stimulation during the day.

The inventor has discovered a balance between the efficacy of circadian phase shift and the potential of being disruptive to sleep. Therefore, the system may include certain light flash parameters to achieve such balance. For example, the inventors have discovered that some combinations of light parameters (e.g., 3000 lux, for 3 ms, once per minute, for 3 hours) have shown efficacy, but can potentially be disruptive for sleep (waking up the users) for certain users. Therefore, for at least some users, the amount of light stimulation is reduced. The inventor has discovered that 750 lux is not disruptive for most people, while still being efficacious in shifting circadian phases.

The current inventor has also discovered that in some cases the current treatment window (3 h) can be too big for some individuals in certain use case scenarios, which causes a reduction in efficacy. Therefore, for at least some users and/or in some use case scenarios, the treatment window can be reduced. Reducing treatment window to the regions with higher confidence on the theoretical Advance or Delay windows on the Phase-response curve, with or without the combination of increasing the flash frequency, could potentially improve efficacy for a wider range of individuals and use case scenarios.

Additionally, shorter light flashes, than the prior light flashes are also effective in shifting circadian phases while causing less disruptions to sleep. It has been found that 10 microsecond flashes, for example, can shift circadian rhythm, and accordingly at least one embodiment of the system provides shorter light flash stimulation than in the prior art.

Therefore, the system may adjust the circadian rhythm at night (or during sleep if the user sleeps at other times of the day) without disrupting the sleep of the users. The system may comprise software, hardware, LED, light calculation, user interface, etc. The light parameters may include any combination of the ranges discussed below.

In at least one embodiment, a personalized programs that produces light pulses (and/or other changes in lighting) based on sleep, travel, work shift information and/or other information related that may affect a user's circadian rhythm. The word “program” may refer to a computer program or other software that the processor runs to turn on and off the light and deliver the treatment plan to the user to adjust and/or regulate the circadian rhythm. The word “program” may also (or alternatively) refer to the treatment plan for adjusting the circadian rhythm (which may be implemented via a software program). Either meaning may be substituted for the word “program” wherever the word “program” appears. The treatment plan may be implemented by software running on a processor, embedded software, firmware, middleware, and/or hardware. The program used for treating the circadian rhythm disorder may be referred to as a light program, when light is used for treating the circadian rhythm disorder or more generally as a circadian rhythm disorder treatment program. Throughout this specification (in both disclosures), the term “circadian rhythm disorder treatment program” and “light program” are used interchangeably—one may be substituted for the other to obtain different embodiments.

In one embodiment, the system may adjust circadian rhythm based on the user's sleep. The circadian rhythm may be adjusted, via an Application Program Interfaces (APIs) of other wearable devices, software applications, and/or through hardware in a sleep mask. The system may include one or several of an 3-6 axis accelerometer, a gyroscope, an activity-monitoring actigraphy, an optical pulse sensor, a respiration sensor (e.g. a microphone inside the system or pulse oximetry by other wearables), and/or an electroencephalogram (EEG) sensors implemented in the sleep mask in contact of the forehead. The EEG may detect brainwaves during sleep, through which the system may analyze to determine the quality and/or stages of the sleep of the user. The system may include sensors like a range finder, a camera, or an electromyograghy (EMG) sensors in the cupped regions around the eye areas of the sleep mask to monitor eye movements during sleep. The system may include a microphone in the sleep mask to monitor sleep by monitoring the user's breathing patterns. The system may include an optical heart rate sensor deployed on the strap of a mask, for example, to monitor heart rate close to the temples to track the heart rate through photoplethysmography. The system may include a temperature sensor to track the body temperature which helps inform the system of the circadian phases during sleep. The system may use one or several of these sensors in combination to track the user's sleep and/or circadian phases in order to achieve a more effective circadian phase adjustment, without disrupting the user's sleep.

Different form factors (e.g., different types of systems of different sizes and shapes and/or using different patterns of light pulses) may be used to deliver the light pulses and adjust circadian rhythms. In an embodiment, the settings of light device is pre-programmed by the manufacturer. In an embodiment, the settings of light device can be adjusted by user input. In an embodiment, the treatment program may be adjusted by the combination of user input and the device calculation based on the data collected. The system may use light pulses of wavelength across 380 nm to 750 nm. The light pulses may have a flash duration of between 1 microsecond-500 milliseconds, which may occur at a periodicity of at least about one flash per minute. The light pulses may occur over a time period between about 30 and 240 minutes carried out during hour 15 to hour 24 (CT15-CT24) or hour 24 to hour 8 (CT24-CT8) of the subject's effective circadian time, part of which may include time periods when the user sleeps and periods when the user is not asleep. To cause the user to wake earlier, the light pulses may occur during CT24 to CT8. To cause the user to wake later, the light pulses may occur during CT15 to CT24. The time CT0 is defined as the cross-over point of the human Phase-response curve or is defined as the time of the fitted minimum of unmasked core body temperature, deducted based on information, such as the user's biological profile, sleep schedules or other information such as core body temperature. In the notation CTxx (where xx is a one-two digit number, as in CT12), CT stands for Circadian Time, and CTxx is defined as the time of activity onset in a free-running human. The effective circadian time is the part of the cycle at which the host's circadian rhythm is currently at. The term “free-running” refers to the conditions under which circadian rhythm is running that are not synchronized to environmental time cues within the normal 24-hour cycle, such as daylight.

Physiological parameters can be captured by sensors in the system to detect where the body is in the sleep cycle and/or the circadian rhythm cycle, and used as input for the circadian rhythm adjustment algorithms. As the user enters stages 1-3 nonREM (N1-N3) sleep, body movement and eye movement decrease, heart rate slows down, breathing slows down, body temperature drops, blood pressure drops, and brainwaves become calmer. During REM sleep, heart rate, respiration, and blood pressure increase, and brainwaves becomes active. During nocturnal awakenings (e.g. a result of the flashes of light being too bright), body movement increases, accompanied by a different eye movement pattern from that in N1-N3 sleep or REM sleep. These parameter changes as the body goes through N1-N3 sleep, REM sleep, and during nocturnal awakenings can be captured to determine where the body is in the sleep state and the circadian rhythm cycle. During a night, the body goes through several sleep cycles (e.g. of 90 minutes to 120 minutes) with different stages. Each of the stages of sleep has a different brainwave frequency and amplitude: waking state/REM sleep: high-frequency (15-60 Hz), low-amplitude activity (˜30 μV); N1: decreased frequency (4-8 Hz), increasing amplitude (50-100 μV); N2: (10-15 Hz, 50-150 μV) spindles; N3: 0.5-4 Hz (100-200 μV). EEG sensors on the head (which may be included in the mask or other elsewhere) can be used to detect the distinct brainwave signature in amplitude and frequency during different stages of sleep. By detecting the brainwaves, the system may be able to recognize which sleep/wake state and/or sleep stages the user is in, and then adjust the circadian clock adjustment program based on the real-time input. For example, the light flash program will be paused temporarily if nocturnal awakenings were detected. Similar parameter changes can be captured by sensors in the system, used independently or in combination, to help inform and adjust the light program. One or more accelerometers (e.g., 3 accelerometers, one for each of 3 perpendicular directions) and/or one or more gyroscopes (e.g., 3 accelerometers, one for each of 3 perpendicular directions) may detect the movement of the body of the user (e.g., the head or the wrist) to determine when the user is in a state of sleep or a state of wakefulness. Respiration rate, which decreases when falling asleep and entering N1-N3 sleep and increases when entering REM sleep, may be detected by sensors detecting sound (e.g. microphone), air flows, oxygen saturation (e.g. pulse oximetry), and/or chest movements. Heart rate, which increases when the user enters REM sleep from nonREM sleep, may be detected by photoplethysmogram (PPG) or pulse oximetry. Eye movement are important for distinguishing between awake, nonREM and REM sleeps. Eye movements may be detected by cameras (e.g. infrared camera) or range finders installed inside the cupped regions of the mask, or through muscle activity sensors around the eyes. Additionally, a camera in the mask may use images taken during the light flashes to determine eye movements. Muscle tones is also an important factors to distinguish sleep/wake state and nonREM/REM sleep stages. Sensors for muscle tones may be installed inside the mask to monitor facial muscle tones or the muscle activities in other parts of the body such as arms or legs. One or several of these sensors and parameters may be used independently or in combination, to detect different states and stages of sleep, which serves as an input for light program adjustments. User input may overwrite the interpretation of the sensors. Given individual's different levels of heart rate, breathing rates, movement patterns and other physiological parameters, boundaries or thresholds that maximize the separation between sleep/wake states will need to be established on an individual bases. The system may store a history of heart rate data, breathing data, brainwave data to establish a base line heart rate, brainwave patterns and breathing patterns, and learn from the data collected by the sensors through, for example, machine learning. By collecting these physiological parameters throughout sleep, the system may detect nocturnal awakenings and/or determine which stage of sleep the user is in, and adjust the light programs accordingly. Optionally, information about daytime activity and light exposure may also be tracked via actigraphy, through which an estimate may be made as to where the user is in the user's circadian rhythm, and determine whether the user's circadian rhythm needs to be adjusted and in which way it needs to be adjusted. Core body temperature is also an important indicator of circadian rhythms and sleep/wake states. Optionally, temperature sensors may be implemented on the body to detect core body temperature directly, or to interpret core body temperature through detecting proximal skin temperature and/or distal skin temperature. Parameters mentioned here may also be captured through other wearables (e.g., wrist-worn tracking wearables) devices. The system of this paragraph may optionally include (or not include) any of the embodiments and/or components of the embodiment disclosed herein.

Optionally, the mask or other wearables may have sensors (e.g. a proximity sensor or a touch sensor) to tell whether or not the mask or the wearable is being worn by the user.

The light parameters may be set and adjusted by the manufacture and/or the user based on user input and/or data captured by the sensors. As an example, the manufacture may set the wavelength of the light or the user may be given a setting for choosing a wavelength of light from wavelength 380 to 750 nm. The light used for treatment may be a mixture of light of different wavelengths from 380 to 750 nm. The light may be white light. The spectrum of colors used to make up the white light may imitate the spectrum of light emitted by the Sun at midday, Sunrise, or Sunset. For example, the light of a red, blue and green LED may be combined, and the percentage of the intensity of three may be chosen to be within 10% of the percentage of the intensity of red, blue and green that are found in Sun light. Additionally or alternatively, the frequency of how often the light flashes may be set to be (by the manufacturer or by the user via a user interface) between at least about one flash per minute, over a time period between about 15 and 240 minutes carried out during CT15-CT24 and CT24-CT8. CT0 may be determined as the cross-over point of the human Phase-response curve or as the time of the fitted minimum of unmasked core body temperature, deducted based on information, such as the user's biological profile and sleep schedules.

The system of this paragraph may optionally include (or not include) any of the embodiments and/or components of the embodiment disclosed herein.

Based on the observations by the inventor as well as researches on human physiology, the light flashes in the system can be shorter than the current duration to the low millisecond or microsecond range, or even nanosecond range (e.g., 1 nanosecond or between about 0.5 and 100 nanosecond), be set by the manufacturer or the user. For example, the light pulse duration may be set by the manufacturer or the user by provided with a setting to result in a light flash of between about 10 microseconds-500 microseconds (e.g., instead of 0.1-2 milliseconds or 1-500 milliseconds) or pulse duration may be set by the manufacturer or the user may be given an adjustable setting as part of the user interface that allows the user to adjust the light pulse duration to result in a light flash duration of between about 10 microseconds −100 microseconds (e.g., instead of 0.1-2 milliseconds, or 1-500 milliseconds, or 10 microseconds-500 microseconds.). Alternatively or additionally, the light pulse duration may be set by the manufacturer. Alternatively or additionally, the user may be provided with a setting for choosing the light pulse duration to result in a light flash of between about 1-25 milliseconds. Alternatively or additionally, the user may be given an adjustable setting as part of the user interface that allows the user to adjust the light pulse duration to result in a light flash duration of between about 1-5 milliseconds.

The system may also suggest or may provide the options to choose different intensity of the flashes. For example, based on the user's light sensitivity and the other properties of the flashes, such as frequency or duration, the manufacturer may provide a default setting of how bright a flash of between about 100 microseconds-1 milliseconds. For example, the flash may have an default intensity of 3000 lux at eyelid level before eyelid penetration set by the manufacturer, with an adjustable range of 500-4000 lux at eyelid level before eyelid penetration.

The user may adjust the light intensity setting to 500 lux or even 250 lux at eyelid level before penetration based on system feedback or the user's own experiencing using the device. The user may adjust other settings that serve as surrogate indicators which result in adjustment of the flash intensity. For example, the user can adjust their light sensitivity setting to change the flash intensity. The system may also automatically adjust the light intensity based on the user's sleep and circadian rhythm shift.

The system may also suggest or provide the options to choose different frequency of how flashes repeat within a certain period of time. For example, the manufacturer may provide a default setting of how frequently a flash of between about 10 microseconds-500 microseconds, or 100 microseconds-1 milliseconds, is repeated over a period of time. For example, the manufacturer or the user may set the flash to any constant value within the range of once per 9 seconds to once per 60 seconds, by default or based on the user's input. Alternatively or additionally, the manufacturer or the user may set the flash to a dynamic value within the range of once per 5 seconds to once per 120 seconds, with optional flash-off periods, to occur for 15 minutes-3.5 hours carried out during CT15-CT24 and CT24-CT8, in which CT0 is defined as the cross-over point of the human Phase-response curve or as the time of the fitted minimum of unmasked core body temperature, deducted based on information, such as the user's biological profile and sleep schedules. For example, the manufacturer or the user may set the flash to occur once every 30 seconds for 60-120 minutes, or once every 15 seconds for 30-150 minutes, or flash-off for 30 minutes then flash-on every 15 seconds for the 30 minutes then flash-off for 30 minutes, carried out during CT15-CT24 and CT24-CT8. The flash frequency can be part of a pre-designed treatment protocol, or responding to the feedback from the users via user input or sensors in the device. The user may be able to change the setting manually or let the system adjust the setting based on the information collected from the device or the app.

The system may also suggest different periods in which the flashes occur based on the user's sleep, travel or shift schedules. For example, the manufacturer may provide a default setting of how long a flash of between about 0.1-0.5 milliseconds (or less), is repeated over a certain chosen period of time, for example. The flash of 0.1-0.5 milliseconds may be chosen to occur at any frequency in the range of once every 9 seconds to once every 120 seconds (e.g., once every minute) for 10 minutes to 180 minutes, for 60 to 180 minutes, or for 30 minutes to 240 minutes carried out during CT15-CT24 and CT24-CT8, in which CT0 is defined as the cross-over point of the human Phase-response curve or as the time of the fitted minimum of unmasked core body temperature, deducted based on information such as the user's biological profile and sleep schedules. Additionally, individuals may be capable of limiting the default settings to narrower ranges (or other ranges) than disclosed above according to what works best for them. The system of this paragraph may optionally include (or not include) any of the embodiments and/or components of the embodiment disclosed herein.

The system may include an application that works together with (e.g., runs on) a mobile device (e.g., a smart phone), other computing device, and/or a circadian rhythm treatment apparatus. An example of a circadian rhythm treatment apparatus is a light pulse delivery apparatus, such as a mobile sleep mask or goggles, and/or other circadian rhythm treatment apparatus (which may deliver light pulses). For example, the system may contain a software and a light-emitting device. The software may be stored on and run on a mobile device (a smart phone), a computational device (a computer), an/or delivered from a server, such as, as a result of the user interacting with a webpage. The software may be located in a light-emitting device (e.g., the sleep mask or a lamp in a bedroom), and the light emitting device may run the program producing the pattern of light used to treat the user. Additionally or alternatively, the light emitting device may respond to signals from a device running the program to produce the pattern of light for treating the user, and thereby the light emitting device may act as the circadian rhythm treatment apparatus. The light emitting device may communicate with a wide area network, such as the Internet, the light emitting device may receive signals, from a server, directing the light emitting device to flash in a desired pattern to adjust a circadian rhythm. The circadian treatment device may be mobile like a sleep mask or a goggle, a bedside lamp, or may not be mobile, such as in-room lighting. The circadian treatment device may have the format of a webpage, which may be part of a downloadable application that may run on a smart phone, mobile mask, tablet, laptop, computer, and/or other mobile device. Alternatively, the circadian treatment device may interface with a sever, or other network-enabled device, without the need to download and/or interact with the webpage. The circadian rhythm device may be located in the cloud and may manipulate another computing device to cause the other computing device to implement the circadian rhythm. For example, a personal computer, workstation, laptop, tablet, or other computing device (e.g., which may be connected to a light) may interact with the a server (e.g., via a webpage) and/or other computing device located in the cloud to cause the user's local computing device to implement the circadian rhythm program, such as causing a light source to flash (the light source may be part of the monitor and/or located elsewhere). The circadian treatment device may deliver light pulses according to a particular timing frequency, color, and/or amplitude pattern. The circadian rhythm treatment apparatus may include a controller (which in turn may include a processor system having one or more processors), memory system, and lights (e.g., an LEDs, fluorescent, and/or incandescent lightbulbs), that may be pulsed or otherwise manipulated according to a pattern that is determined by the mobile application, via the controller. In any of the embodiments, the light flashes can be received either through the eyelids (of closed eyes) when the user is sleeping or resting, or directly into open eyes when the user is awake during the night or within 1-2 hours before or after sleep. Some users may, at times, sleep with their eyes open, some users may wake up in the middle of sleeping, and some users may at times rest their eyes in the middle of the day without going to sleep. Accordingly, in an embodiment, prior to applying a given flash or prior to applying a given set of flashes, a sensor may detect whether the user's eyes are open or closed and apply the appropriate amount of light depending on whether the eyes are open or closed regardless of whether the user may be in fact sleeping. Optionally, while a user is sleeping, if during the course of a flash it is determined that the user's eyes are open, the intensity of the next flash may be lowered to an intensity appropriate for when the user's eyes are open. The intensity of the lights will be adjusted based on whether or not they have to penetrate through the eyelids.

In an embodiment in which the system includes a mobile device and mobile application, the mobile application and the mobile device may work together. For example, the mobile application may cause a user interface to be presented to the user. In response, the mobile device receives input (e.g., entered by the user) that indicates the user's sleep schedules, travel information, work shift information, and/or other circadian rhythm information (although a mobile device is often referred, the mobile device is just one non-limiting example. The application may run on a stationary device, website, and/or in the cloud, for example). Based on the input information, a model to adjust the user's circadian rhythm and make the user's circadian rhythm fit the user's schedule is computed, and a therapeutic treatment (or preventative treatment) is computed. Throughout the specification (in both this specification and the appendix), the term therapeutic treatment is used interchangeably with preventative treatment, wherever either term appears one may be substituted for the other to obtain another embodiment. The treatment may be a pattern of light pulses and/or other changes in lighting that are delivered to the user, via the circadian rhythm treatment apparatus that delivers the treatment (e.g., via a program that controls the lighting, such as by controlling pulses of light delivered to the user) to effectively treat or prevent circadian rhythm disorders. The treatment may be applied while the subject is asleep and/or awake in the middle of sleep or within 1-2 hours before or after sleep.

In one embodiment of the circadian rhythm treatment, the circadian rhythm treatment apparatus can be an eye mask that the user wears while sleeping, for example. The mask may work with the mobile application. The mask may use a personalized circadian rhythm disorder treatment program (e.g., a personalized light pulse program or other circadian rhythm disorder treatment program) to treat or prevent circadian rhythm disorders. The program may run on a mobile phone, mobile device, computing device, and/or a server (e.g., the user may interact with a server-based program, via a webpages). In other embodiments, other forms of light treatment may be used, such as goggles, a hood, a screen, or a lamp, for example.

In one embodiment, the circadian rhythm treatment apparatus may adjust circadian rhythm based on the user's current sleep schedules as compared to a desired sleep schedules, or based on the user's desired work schedules, where the user may input information indicating the change in the sleep pattern desired, via a graphical user interface. Optionally, the circadian rhythm treatment apparatus may have an Application Program Interface (API), via which another device may control or interface with the circadian rhythm treatment apparatus, allowing the user to adjust the user's sleep pattern via the API. The circadian rhythm treatment device may communicate with other devices and/or programs via the API associated with those other devices and/or programs. The circadian rhythm treatment apparatus may interface with a wearable device and/or software applications, or through a program and/or hardware device that controls the collection of Internet of Things (IoT) devices (e.g. a smart home device or a virtual intelligent personal assistant). Alternatively or additionally, circadian rhythm treatment apparatus may be incorporated into a wearable devices and/or software applications.

In one embodiment, the circadian rhythm treatment apparatus may interface with and/or include a sleep mask that includes sensors, which may detect the user's current circadian phase and/or sleep stages and determine a treatment and/or adjustments to a treatment to achieve a desired sleep pattern. For example, the circadian rhythm treatment apparatus may include one or more accelerometers and/or gyroscope, one or more motion-monitoring actigraphy, and/or one or more EEG, eye movement sensors, respiration and/or pulse sensors as sensors in the mask. The sensors and/or other electronics may be located in the sleep mask directly or indirectly in contact with the forehead and/or in a region that where eye movement can be detected (e.g., optically), such as in the portion of the mask that covers the eyes. Optionally, the sensors and/or other electronics may be located on other places, for example, inside the mobile phone, under the mattress, or on the user's wrist or chest. The actigraphy sensor may monitor circadian phase and sleep patterns. In this specification, the terms actigraphy and actimetry sensor are used interchangeably and can be substituted one for another to obtain different embodiments. The actigraphy may measure gross motor activity (e.g., that is indicative of sleep patterns). Optionally, the actigraphy may be worn on the wrist and/or-may be shaped similar to a watch. Optionally, the movements sensed by any of the sensors (e.g., which result from the user's movement while sleeping/resting) may be recorded. Optionally, the actigraphy and/or other sensors may measure light exposure. Optionally, the actigraphy and/or other sensors may have an interface for attaching to a computing device in addition to, or instead of, the interface of the circadian rhythm treatment apparatus. The computing device may analyze data (optionally in real time) in addition to, or instead of, the circadian rhythm treatment apparatus analyzing the sleep data.

Optionally, one or more of the sensors of the circadian rhythm treatment apparatus may be range finders or cameras, which may be placed in the cupped eyeholes of the mask to monitor eye movements during sleep. Optionally, one or more of the sensors of the circadian rhythm treatment apparatus may include a microphone, which may be located in the sleep mask to monitor sleep through user's breathing patterns. Optionally, one or more of the sensors of the circadian rhythm treatment apparatus may include one or more heart rate sensors. For example, the circadian rhythm treatment apparatus may include an optical heart rate sensor to track heart rate through photoplethysmography. The circadian rhythm treatment apparatus may include one or more temperature sensors to track the body temperature (and or atmospheric temperature). Tracking the body temperature may help inform the user's circadian phases and sleep stages. The circadian rhythm treatment apparatus may include one or more sensors for muscle tones and activities, implemented inside the mask or located on other parts of the body. Optionally, the optical heart rate sensor and the motion sensors may be worn on the wrist. The circadian rhythm treatment apparatus may include any one or any combination of these sensors in combination to track the user's sleep. The circadian rhythm treatment apparatus may also access the API of other wearable devices to obtain sleep and circadian data. The sleep/wake status and the circadian phases calculated from data obtained by such sensors and devices may be used by the circadian rhythm treatment apparatus to achieve more effective circadian phase adjustment while preventing disrupting user's sleep.

Any of the examples of circadian rhythm systems (in either disclosure) may be substituted one for another and/or used anywhere in the specification to obtain new embodiments.

Any of the above embodiments may be used alone or together with one another in any combination. Inventions encompassed within this specification may also include embodiments that are only partially mentioned or alluded to or are not mentioned or alluded to at all in the in the above discussion.

FIG. 1 shows an embodiment of system 100 in which the circadian rhythm treatment hardware is built into a sleep mask. Mask 102 covers the users eyes and blocks environmental light from shining on the eyes of the user. Mask 102 may be fashioned from a soft and/or resilient material. Cupped regions 104a and 104b are regions that form cups away from the user's eyes, to ensure that the mask does not cause discomfort by pressing against the user's eyes and/or to ensure that the lights that generate the flashes are a predetermined distance from the eyes to ensure proper illuminaiton of the eyes during a light flash. Lights 106a and 106b are regions where lights and/or light pipes are placed for illuminating the user's eyes with light flashes. Lights 106a and 106b may be located in cupped regions 104a and 104b. Straps 108a, 108b, 108c, and 108d hold mask 102 on the user's head. The mask may have sensors on the forehead, the cupped eye regions, and/or the straps to collect data on motion, heart-rate, sound, and/or temperature.

FIG. 2 shows an embodiment of system 200 in which the circadian rhythm treatment hardware is built into the frames. System 200 may include eyepieces 202a and 202b, lights 204a-d, bridge 206, and/or arms 208a and b.

System 200 may be a pair of glasses, which may be used within 1-2 hours before or after sleep, for example. System 200 may include eyepieces 202a and 202b, which may include transparent lenses or an opaque material for use while napping. In an embodiment in which 202a and 202b are lenses, the lenses of eye pieces 202a and 202b may be prescription lenses or nonprescription lenses. Lights 204a-d are lights for treating the user (e.g., to adjust the user's circadian rhythm. Lights 204a and b are located in lens 202a and lights 204c and d are located in lens 202b. Lights 204a-d may be similar to, or identical to, lights 106a and b. However, the components of lights 204a-d may need to be located, sized, and/or or structured so as to minimize obstructions to the user's vision. For example, the components of lights 204a-d that are located in eyepieces 202a and b may be transparent and/or small enough to not be noticeable. Also, the index of refraction of the lenses of eyepieces 202a and b and the index of refraction of the components of lights 204a-d may be chosen to minimize internal reflections, so as to help minimize the degree to which lights 202a-d obstruct the vision of the user. Using two lights in each lens allows for a more uniform illumination of the eyes, thereby allowing for a greater region of illumination with a lower intensity of light. Having two lights in each eyepiece is optional. In other embodiments, there may be just one light in each eyepiece or thereby may be more than one light in each eyepiece. Bridges 206 hold eye pieces 202a and 202b attached to one another. Arms 208a and b are attached to the eye pieces 202a and b, and hold system 200 on the user by resting on the portion of the user's ears that are attached to the user's head. The combination of bridge 206 and arms 208a and b hold the eye pieces 202a and b in front of the user's eyes, with bridge 206 resting on the user's nose, so that lights 204a-d may properly illuminate the user's eyes during treatment. In an alternative embodiment, eye pieces 202a and b may be opaque or translucent and system 200 may be worn while resting.

In an alternative embodiment, some or all of the hardware may be built into the lenses (of FIG. 2).

The mask of the embodiment of FIG. 1 or eyeglasses or frame of the embodiment of FIG. 2 may work with a mobile application and may use personalized light pulse program to treat and/or prevent circadian rhythm disorders.

FIG. 3 shows an embodiment of system 300. System 300 may include ceiling lights 302a and b, table lamps 304a and b, furniture 306, blankets 308, and/or pillows 310a and b.

System 300 has circadian rhythm treatment hardware that is built into a lighting system of the room of a building (e.g., a bedroom). Ceiling lights 302a and b are light that are attached to, or that hang from, the ceiling and/or wall. Ceiling lights 302a and b may be used to deliver a circadian rhythm treatment (and to illuminate the room). Although two ceiling lights are shown in the embodiment of system 300, there may be one ceiling light—any number of ceiling lights may be used. Table lamps 304a and b are lights that may rest on the furniture or floor, and may be used to deliver a circadian rhythm treatment (and to illuminate the room). Furniture 306 may be used by the user while receiving the circadian rhythm treatment. Blanket 308 and/or pillows 310a and b may be used by the user while receiving the circadian rhythm treatment. In an embodiment, the user may receive the circadian rhythm treatment while sleeping or relaxing on bed 306 (or in a chair), via ceiling lamp 302a, ceiling lamp 302b, table lamp 304a, table lamp 304b and/or other lamps or lights. In an embodiment, the user may receive circadian rhythm treatment while awake and performing other activities within 1-2 hours before or after sleep, or in the middle of sleep in case the user wakes up, via ceiling lamp 302a, ceiling lamp 302b, table lamp 304a, table lamp 304b, and/or other lights, in addition to and/or instead of receiving the treatment while sleeping or relaxing. Optionally, bed 306, Blanket 308 and/or pillow 310a, and/or pillow 310b may be replaced with other furniture and/or items. Another embodiment of the circadian rhythm treatment apparatus may be built into, part of, and/or include a lamp or a set of lamps (controlled by a controller), that works with a mobile application and uses a personalized circadian rhythm disorder treatment program (e.g., light pulse program) to treat or prevent circadian rhythm disorders. Motion sensors and environmental sensors may be embedded in 302, 304, 306, 308, and/or 310 to collect sleep data. 302, 304, 306, 308, and/or 310 can also be existing hardware with sleep-tracking functions, whose data may be access by the circadian rhythm treatment program through API in order to adjust circadian rhythm more effectively without being disruptive to user's sleep. The circadian rhythm treatment program may also work through accessing the API of a program and/or hardware device (an example of such a hardware device is discussed below in conjunction with FIG. 5) that controls the collection of Internet of Things (IoT) devices including the ceiling lamp 302a, ceiling lamp 302b, table lamp 304a, table lamp 304b and/or other lamps or lights.

The circadian rhythm treatment system may also deliver treatment via a computer display, television display, game console display, or mobile device display, which may be used for illuminating a confined area while the user is sleeping in involved in another activity (not related to the computer display, television display, game console display, or mobile device display. In an embodiment, the pulses of light used for circadian rhythm treatment system are too bright to be comfortable if applied while directly watching screen providing the light. However, the user is free to use the circadian rhythm treatment while the person is working on the computer or using the mobile device, watching television, and/or playing a video game, anyways, should the use choose to do so. The circadian rhythm treatment program may run on a mobile device (e.g., a cell phone or laptop) that is left on while the user is sleeping or engaged in other activities (but no looking directly at the screen).

Another embodiment of the circadian rhythm treatment apparatus may include in-room lighting (controlled by a controller) that works with a mobile application and uses a circadian rhythm disorder treatment program (or personalized light pulse program) to treat or prevent circadian rhythm disorders.

The circadian rhythm treatment system may be built into any lighting system, such as in any part of a home, television, vehicle, and/or outdoor area, in which the circadian rhythm treatment hardware is built into a lighting system of the room of a building (e.g., a bedroom). The circadian rhythm treatment system may be built into any lighting system, such as in any part of a home, television, vehicle, and/or outdoor area.

### Light Settings

The circadian rhythm treatment system can be used before, during, and/or after the occurrence of the change of sleep schedules or time zones or anytime when the user would like to improve sleep through optimizing circadian rhythms. Different programs may be established, presented, or started when the user starts the program depending on the time when the user open the circadian rhythm application.

For example, if traveling from San Francisco to New York, which are 3 time zones apart, the user may use the any of the system of FIGS. 1-3 the night before the flight and arrive without jet lag. For example, if traveling from San Francisco to Beijing, which are about 9 time zones apart, the user may use any of the system of FIGS. 1-3 (e.g., possibly the lights of FIG. 3) the night before the flight, and possibly the mask of FIG. 1 and/or the goggles of FIG. 2, 1-2 nights after landing to reduce the effects of the change in times zone.

The circadian rhythm treatment systems of FIGS. 1-3 use different light pulse settings such as wavelength of the light, duration of pulses of light, interval of time during which pulses of light are delivered, the frequency of the pulses, the timing, and duration of the circadian rhythm disorder treatment program (e.g., via the light program).

For example, depending on the light sensitivity of the user, pulses of different intensity may be delivered to effectively treat the user (treating or preventing circadian rhythm disorder) without waking up the user.

As part of the circadian-rhythm-disorder-treatment-program, a light-pulse-program may be used alone or in combination with a continuous light program before sleep or towards the end of sleep. The continuous light program may accompany and/or complement a circadian rhythm program, which may have different uses, settings, such as wavelength of the light, the timing of the light, and the duration of the light treatment. Optionally, the user may use the continuous light program, by itself, without the circadian rhythm treatment program. For example, the continuous light program may be used for morning wake up, or may be supportive of an audio for meditation, which may be used with or without a circadian rhythm program in which a pattern of light pulses is delivered.

For example, exposure to bright continuous light in the morning may stimulate the secretion of cortisol, and activate the sympathetic neural system, preparing the body to get ready for the day. In an embodiment, the user can choose different wavelengths of the light, different timings, and different durations of treatment administered by the mobile interface in the circadian rhythm disorder treatment program.

There may be an audio component to the circadian rhythm disorder treatment program, which may produce therapeutic sounds, relaxing sounds, and/or pleasant sounds that are synchronized with, and played together with, or independent of, the treatment, such as the pattern of light produced.

### Program

The mobile application uses the user's personal parameters such as the sleep schedules and sleep stages, travel, (optionally current sleep) and/or sleep shift schedules, as well as some biological parameters, and optionally, information about specific applications such as Daylight saving time or seasonal affective disorders (SAD), to calculate a personalized circadian rhythm disorder treatment program (e.g., a light pulse program) to treat or prevent circadian rhythm disorders.

The mobile application takes input both from the user and from the circadian rhythm treatment system, which is used to calculate the circadian rhythm disorder treatment program (e.g., light therapy program), then the program is sent to the circadian rhythm treatment apparatus, and the circadian rhythm treatment apparatus delivers the treatment automatically and/or when activated.

For example, the user may input their normal sleep schedules (bedtime, wake time, etc.) into an interface presented by the application that implements the circadian rhythm therapy. The application may also receive user-input that includes sleep schedule changes. For example, the user may plan to work a second 8-hour shift for a week then adjust back to the user's usual schedule. The user may enter input into the interface, indicating that the user will be working the 8-hour shift, when the user will be working the 8-hour shift, and/or when the user expects to sleep. Then the application may calculate the treatment program based on the user's needs and deliver a circadian rhythm therapy program that shifts the user's circadian rhythm to accommodate the new schedule.

As the user uses the circadian therapy program, more data on the changes and response to circadian rhythm shift may be gathered to establish a more accurate baseline, and the application will incorporate the data into computation of the circadian rhythm disorder treatment program (e.g., light program).

The light therapy program is dynamic and can be improved based on additional information collected each day from the user or from the circadian rhythm treatment apparatus.

In an embodiment, there are sleep sensors in the mask that track the sleep schedule and sleep/wake or stages of sleep of the user, such as by detecting the baseline of relevant physiological parameters, whether the user's eyes are closed, the user's head and body movements, the user's heart rate, and/or breathing pattern. The mask may first establish a baseline shift program based on the average shift that can be achieved on a populational level, and then the application (which may be an embodiment of the system of FIGS. 4A) may adjust itself based on the sleep time of the user once the program starts. For example, if the mask determines that the user has achieved the entire shift in the user's circadian rhythm earlier than the populational average time for achieving the same shift in circadian rhythm, then the program may stop. The user can also stop the program manually based on how adjusted they feel.

In an embodiment, the user can input multiple schedules at one time. The program then may automatically detect and automatically generate a program to accommodate all the schedules to implement at a given time based on calendar information input into a calendar and/or GPS information.

In an embodiment, multiple users may use the same device at different times. Each user may have his/her own account with past programs, biological, sleep, and behavioral profiles. Based on the information collected throughout the time that user uses the circadian rhythm treatment apparatus, the software adjusts and improves itself to provide personalized circadian rhythm disorder treatment program (e.g., light program).

Another embodiment of the software can be a web-based or computer-based application that serves the same or similar purpose as the application that runs on the mobile device.

There may be an audio component works together with the circadian rhythm disorder treatment program (e.g., light program).

### Wavelength

In an embodiment, the flashes are white light emitted from LED that covers the full spectrum or a subset of the full spectrum of the visible light (380 nm to 750 nm). In an embodiment, the light flashes have a mix of wavelengths between 380 nm to 750 nm. In an embodiment, the flash can use white light emitted from other light-emitting components that covers the full spectrum or a subset of the full spectrum of the visible light. The morning or evening continuous light that is independent of the circadian rhythm-adjusting light flashes may have a different wavelength range than the white flashes.

FIG. 4B shows an example of a treatment 400 regimen for treating circadian rhythm, which may include treatment periods T1 and T2, which include series of flashes, having width W, in which the flashes may have a duration D and period P, which are of an intensity of Lf_{open-eyes} or Lf_{closed-eyes}. The treatment periods T1 and T2 may each be blocks of flashes, T1 is the time throughout the duration of which the block B1 may happen, and T2 is the time throughout the duration of which the block B2 may happen. T1 and T2 may represent separate treatment windows in which circadian rhythms are delay or advanced. Each block (B1 and B2) may divided into subblocks. For example, block B1 may include subblocks b11, b12, b13, . . . , and block B2 may include subblocks b21, b22, b23, . . . . The period P is the time between a particular point in the cycle of the turning the flash on and then off or off and then on (e.g., the start of one flash) and the point on the next cycle (e.g., the start of the subsequent flash). The frequency of the flashes may be computed from the period, P, according to the formula f=1/P. T1, T2, W, P, f, and D are discussed further below. Although in the example of FIG. 4B only one light pattern with one width W and one duration D were shown distributed homogenously throughout treatment windows T1 and T2, there may be more than one light patterns with different W, D, and f distributed heterogeneously within treatment windows T1 or T2. The term “width” refers to a period of time. The width of a flash refers to the period of time of the duration of the flashes—that is how long the flash lasts, as opposed to the period of time that the flash is turned on, because after the flash is turned off the flash may last for a short while longer, and the time that the light continues to glow after being turned off is included in the duration of the flash. The width of a train of flashes, refers to a period of time over which a series of flashes occurs, and the width of a treatment period is the duration of time over which a treatment period occurs. In each treatment period there may be multiple widths, each for a different series of flashes or sets of series of flashes, and the flashes within a given series of flashes may have different durations or different sets of durations. In other words, the widths of the flashes and/or the series of flashes (which may be referred to as a block of flashes) within one treatment period may have one width or one set of widths and the widths of another treatment period (the flashes or train of flashes or block of flashes) may have another width or another set of widths. In this specification, the term “block,” as in a block of flashes, refers to a “train” of flashes. The terms a “block of flashes” and a “train of flashes” are used interchangeably throughout this specification, and may be substituted one for another to obtain different embodiments. However, the term “block” may also refer to a block without any flashes. Similarly, the flashes within one series of flashes (or train of flashes) may have one duration or one set of durations and the flashes in another train of flashes may be another duration or another set of durations.

Lf_{open-eyes} is the intensity of light of the flashes that is applied by the system of an embodiment of FIGS. 1-3 when the user's eyes are open, and Lf_{closed-eyes} is the intensity of light that is applied when the user's eyes are closed. A higher intensity is applied when the user's eyes are closed than when the eyes are open. Lf_{open-eyes} and Lf_{closed-eyes} are chosen so as to be high enough to achieve efficacy, but low enough so as to not be uncomfortable. Lf_{open-eyes} and Lf_{closed-eyes} may be initially set to default values based on an intensity that an average individual finds comfortable and still results in an effective treatment. The user may later adjust the settings to what works best for that individual. Alternatively or additionally, the values Lf_{open-eyes} and Lf_{closed-eyes} of may be adjusted automatically based on sensor reading. FIGS. 4C-4E shows an example of one or more blocks of light flashes having a block width W within a treatment window. One treatment window may contain one or more light flash blocks, and each block contains the same light flash patterns that may have one set of light parameters such as intensity, duration, and frequency, or a regular repeat of flashes with multiple sets of different parameters. The blocks may be adjacent to each other, or separated by a block of time when no light flashes occur. In an embodiment, the width of a single block may be between 10 mins to 3 hours, but the total width of all the blocks within a treatment window do not exceed the 3.5 hours. The frequency f1 of the flashes during the block of flashes may be varied. Although the flashes of FIG. 4C have a frequency of f1= 1/9 second to 1/120 seconds (about 0.11 Hz to about 8.3 millHz), other frequencies may be used. Although in the example of FIG. 4C, only one frequency is used for the series of flashes, in other embodiments the frequency of flashes may be varied during the series of flashes.

FIG. 4D and E show an examples of two consecutive series of flashes 440 and 460, in which each block of flashes is of a different frequency. In FIG. 4D, the two consecutive blocks of flashes 440 (which are labeled B1 and B3) are spaced apart by a period of time (e.g., 10 minutes to 3 hours by a block B2, which is a block with no flashes), whereas in FIG. 4E the two consecutive blocks of flashes are back-to-back with no period of time between them. Although in the example of FIG. 4D and E, each block of flashes has the same width W, each block of flashes may have a different width. Although each block of flashes of FIGS. 4D and E has a constant frequency, as mentioned in conjunction with FIG. C, each may have a varying frequency, and the manner in which the frequencies vary may be a different manner. Although in the example of FIG. 4D and E each block of flashes has the same intensity Lf, each block of flashes may have a different intensity.

Intensity (Lf_{open-eyes} and Lf_{closed-eyes}, FIG. 4B)

In an embodiment, intensity of the flash, Lf_{closed-eyes}, may be limited to being at least 250 lux or within the range of 500-3500 lux measured at the level of eyelid before penetrating through the eyelid of the closed eye. In an embodiment in which the flash duration is between about 1-5 milliseconds (e.g., 3 millisecond), the intensity of the flash may be 750-3000 lux as measured at the level of eyelid before penetrating through the eyelid. In an embodiment, the intensity of the flash may be 1000 lux or higher, such as around 1500-2500 lux measured at the level of eyelid before penetrating through the eyelid. In an embodiment in which the flash duration is less than 1 milliseconds, the intensity may be 1000 lux or higher, such as, as high as 4000 or 5000 lux, measured at the level of eyelid before penetrating through the eyelid. In an embodiment in which the user is sleeping or resting with closed eyes or awake (or sleeping) with open eyes, the intensity may be at least 25 lux, 100-1000, or 50-500, or 100-5000 lux measured at the level of the open eye or at the level of eyelid before penetrating through the eyelids so the user has the flexibility of adjusting based on their sleep/wake situation. In the embodiments which apply when the user is sleeping or resting with closed eyes, the light intensity of flashes is determined partially based on the eyelid attenuation assumption used in some sleep research, in which on average about 10% full-spectrum light penetrates through the eyelid. Compared to using sensors to measure attenuation and then to determine the flash intensity, this assumption simplifies the device design, avoid components that are potentially uncomfortable for the user's eyes, and prolongs the battery life of the device. Thus, although often in this specification, the intensity for Lf_{closed-eyes} is given without expressly specifying Lf_{open-eyes}, the corresponding value or range of values of Lf_{open-eyes}, is given by Lf_{open-eyes}=(Lf_{closed-eyes})/10. Similarly, when in this specification, the intensity for Lf_{open-eyes} is given without expressly specifying Lf_{closed-eyes}, the corresponding value or range of values of Lf_{closed-eye}, is given by Lf_{closed-eye}=10(Lf_{open-eyes}).

### Duration (D, FIG. 4B)

How long a single flash lasts is referred to as the duration of flash, D. The time span (W) of a block (B1, B2) of light flashes, subblock (b11, b12, b13, . . . , b21, b22, b23, . . . ) of light flashes, and/or of each of the light flashes may vary depends on the user's sleep, travel, and shift information, as well as some of the user's biological parameters. In an embodiment, the duration of a subblock of flashes is Wsub, if the subblock has three flashes of duration D, each followed by a period of time with no flash of duration D′, if D is 3 milliseconds and D′ is 10 seconds, the sublock is 30 seconds and 15 milliseconds. In an embodiment, the duration of each flash (e.g., D) is less than 3 milliseconds. In an embodiment, the flash duration (e.g., D) is 10 microseconds or higher. In an embodiment, the flash duration (e.g., D) may be 10 microsecond to 500 microseconds, 100 microseconds to 1 millisecond, 500 microsecond to 3 millisecond, or 10 microseconds to 10 millisecond. In an embodiment, the flash duration (e.g., D) maybe 1 microsecond to 500 milliseconds. In an embodiment, the flash duration (e.g., D) maybe 1 nanosecond to 1 microsecond.

### Frequency (f1 and f2, FIGS. 4C-E)

In an embodiment, the frequency f1 is once per minute or higher. In an embodiment, the frequency is once every 9 seconds or lower. In an embodiment, the frequency of the flash is limited to once every 9 seconds to once every minute, for example, once every 30 seconds. In an embodiment, the frequency (f1) is limited to once per 5 second to once per 90 seconds. In an embodiment, the frequency is limited to once per second to once per 120 seconds. The flash frequency may be a constant number throughout the course of treatment, or can change throughout the course of the treatment as part of a pre-designed treatment protocol, or responding to the feedback from the users via user input or sensors in the device. For example, the system may cause there to be no flash for a first block of time B1, and then the block, B1, with no flash may be followed by flashes at a frequency of once for 9-60 seconds (for example, once every 15 seconds) for a second block of time, B2. Alternatively, there may be no flash for a period of time B1 followed by a train of flashes at a frequency of (f1) once per 9-60 seconds (for example, once every 30 seconds) for a period of time B2 (as in the prior example), which may then be followed by no flash for a third block of time B3. Alternatively, the second period of time B2 may have flashes at a frequency of once per 9-60 seconds, may be followed by a third period of time B3 with flashes at a frequency (f2) of once per 30-120 (or 30-60, or 60-120) seconds, for example, once every 90 seconds. The period of time B3 having flashes once per 30-120 seconds may be followed by another (fourth) period of time B4 having flashes once per 9-30 seconds for a period of time, then followed by a fifth period of time B5 having flashes once per 30-120 (or 30-60, or 60-120) seconds or flash off for a period of time, are also within the scope of this specification.

### Treatment Window (T1 and T2 FIG. 4B)

In an embodiment, the flashes are delivered once per minute for 3 hours carried out during hour 15 to hour 24 (CT15-CT24) (which may be T1) or hour 24 to hour 8 (CT24-CT8) (which may be T2) of the subject's effective circadian time, in which CT0 is defined as the cross-over point of the human Phase-response curve or as the time of the fitted minimum of unmasked core body temperature, deducted based on information, such as the user's biological profile and sleep schedules. For usage in an embodiment of systems of FIGS. 1-3, CT0 is defined as, may be determined by measuring, the time of the fitted minimum of unmasked core body temperature, which may derived from information, such as the user's biological profile and sleep schedules (CTO may be inferred from the pattern of the user's body temperature). In another embodiment, the flashes may be delivered at either a constant frequency within the range of once per 5 seconds to once per 120 seconds, or a changing frequency within the same range. The system may switch the pulses on-and-off, delivered in the treatment window of anywhere within the range of 10 minutes to 3.5 hours during hour 15 to hour 24 (CT15-CT24) or hour 24 to hour 8 (CT24-CT8) of the subject's effective circadian time.

In an embodiment, any of the frequencies disclosed in this specification may be used, and may be applied for 30 minutes off, 30 minutes on, and then 30 minutes off or for another number of minutes on and off. In an embodiment, when turning the flashes on for a period and then turning the flashes off for another period of time, the total number of minutes to treatment (the sum of Ws of all the blocks B with light flashes) may be limited to 5 minutes to 3.5 hours. In an embodiment, the on/off periods are not necessarily equal. In an embodiment, the on-periods are longer than the off periods. In an embodiment, the off-periods are longer than the on-periods. In an embodiment, the on-period proceeds the off-period, which in another embodiment, the off-period proceeds the on-period. In an embodiment, there may be several cycles of on/off periods. In an embodiment, the on and off periods is determined by the sleep/wake and/or sleep stages of the user, based on theoretical analysis and/or data captured by the sensors.

In an embodiment, the frequency is automatically varied (to create “dynamic” frequency treatment) during the treatment period, based on sleep/wake and/or sleep stages computed using data collected from sensor readings. For example, there may be a 30 minute period in which the frequency of the flash is once every 60 seconds, followed by 30 minute period having flashes of a frequency of once every 10 seconds, and then followed by 30 minute period with flashes having a frequency of once every 90 seconds. Optionally, there may be a period of no flashes between each of the period of flashes during the dynamic frequency treatment. According to how the user sleeps at different times, there are an unlimited number of combinations of frequencies during different periods and the durations of each period have any given frequency. Optionally, the length of periods (blocks) with light flashes and without light flashes and the flash frequency within each block can be dynamically changed, determined in real time while the user is sleeping and receiving the circadian adjustment treatment, based on data captured by the sensors. In an embodiment, the total treatment period is at least 5 minutes long. In an embodiment, the treatment period is 240 minutes long or less.

Any frequency or range of frequencies may be used with any intensity or range of intensities, any color or range of wave length of color, any flash duration or range of flash duration, treatment period or range of treatment periods, every treatment pattern of changes in frequency and/or on/off periods may be used together with one another.

In an embodiment, the values of parameters have a tolerance of 10% of the value specified. Dynamic circadian rhythm adjustment based on user's sleep.

### Program

FIG. 4A shows a flowchart 400 of an embodiment of a method of operating the system of FIGS. 1-3. In step 402, user input is received from which a treatment programs is determined. Steps 404-412 and 420 are substeps of step 402. In step 404 a biological profile is determined. The biological profile may include biological information that is relevant to the user's sleep pattern. The biological profile may include the user's age, sex, weight, height, amount of exercise, whether or not the user has insomnia, sleep apnea, a heart condition, brain malady, a respiratory condition, or other sleep disorder and/or other biological issue that may affect the user's sleep pattern.

In step 406, information about the user's sleep profile is collected, which may include the user's normal bedtime and wake time, the number of hours the user ordinarily sleeps, the number of times the user gets up in the night, whether the user has a form of insomnia, sleep apnea, or other sleep disorder.

In step 408, a long-term profile is assembled from the biological profile and sleep profile. In step 410, user input is received. The user inputs may include information regarding a desired circadian rhythm, in the format of upcoming, current, or past travel or shift schedules, or desired new wake time and/or new bedtime. For example, the use inputs information about an upcoming trip, an expected sleep schedule and/or change in schedule. The user may also treat the circadian misalignment resulted from a trip or shift schedule that is ongoing or just concluded. In step 412, the treatment plan is determined based on the long-term profile of step 408, and the user input of step 410.

The treatment (which was determined in step 412) is implemented and data may be collected and used to adjust the treatment program. Step 414 represents the data collection by the circadian adjusting device, for example, circadian shift progress and/or quality of sleep, and the program adjustment. Steps 416 and 418 are substeps of step 414. In step 416, an adjusted sleep treatment program is implemented. Initially, the adjusted treatment program of step 416 and the treatment program 412 are the same program. As the sleep program is running and sensor information is received, the treatment program is adjusted based on sensor information received. The adjustment to the treatment program may occur in the middle of a treatment program and/or may be applied to a future treatment. In step 418, sensor information is collected, which is used to adjust the treatment. In step 420, user input is received based on the user's experience, which is used to adjust a current or future treatment. Optionally, the user input of step 420 may override the input from step 418. Alternatively or additionally, the user input of step 420 may be used to check whether the adjustments made automatically during step 418 improved the user's perception of comfort and/or the user's perception of the effectiveness of the treatment. Alternatively or additionally, based on the user input of step 420 further adjustments may be made to the treatment regimen, in addition to or instead of the adjustments of step 418. Alternatively or additionally, the user interface may provide information about whether the program is functioning properly and/or properly aligned. For example, the application may provide a message indicating successful completion or what needs to be completed and/or a progress bar indicating progress in setting up the program.

### Hardware

FIG. 5 shows a block diagram of an embodiment of a computing system 500. Computing system 500 may include computing device 501, an application 502 and display 506. Computing system 500 may also include cloud 508 and/or circadian rhythm adjustment device 510.

FIG. 5 shows a block diagram of an embodiment of the circadian rhythm therapy system, computing system 500. Computing device 501 may be a mobile device, Personal Computer (PC), laptop, smart phone, and/or other computing device. Application 502 may reconfigure a computing device 501 to function as a circadian rhythm adjustment apparatus and/or may interface with a circadian rhythm adjustment apparatus. Display 506 may be a touch screen or other display via which the user may view setting and pages generated by application 502 and enter input for configuring the computing device 501 and/or systems 100, 200, and/or 300 for adjusting a circadian rhythm. Cloud 508 may include any combination of wide area networks and/or local area networks. Cloud 508 may include one or more servers, computing devices, and/or devices on which an algorithm for determining a circadian rhythm therapy and/or controlling sleep therapy system may reside. Circadian rhythm adjustment device 510 may be any of system 100, 200, or 300. Circadian rhythm adjustment device 510 may be controlled directly by computing system 501 and/or a device on cloud 508. Besides storing the data collected from the mobile application 502 and/or from circadian rhythm adjustment device 510, the cloud 508 may run application 502 and/or may run an algorithm based on input from application 502 (which is running of computing device 501), and cloud may send the sleep therapy program to computing device 501 and/or circadian rhythm adjustment device 510. Computing device 501 may communicate with circadian rhythm adjustment device 510, via cloud 508.

FIG. 6 shows a block diagram of a system 600 for adjusting a circadian rhythm. System 600 may include power 602, near field communications 604, environment sensors 606, sleep sensors 608a. Sleep sensor 608a may include temperature sensor 608b, pulse sensor 608c, eye motion sensor 608d, body motion sensor 608e, breathing sensor 608f, microphone 608g, and/or brainwave detector 608h. System 600 may include light system 610, which may include left light 612 and/or right light 614. System 600 includes microprocessor 616a, pulse modulator 616b, treatment logic 616c, brightness control 616d, sound system 618, which includes an optional microphone 620 and speaker 622, other communications 624, and/or other components 626.

The circadian rhythm treatment apparatus (e.g., systems 100-300, computing device 501, and/or system 600) may contain a power source (e.g., battery, adapter, and/or power cord), a Bluetooth chip, radio frequency communications chip, Wifi chip, nearfield communications chip that connects directly or via a Wide Area Network (e.g., the Internet). Alternatively or additionally, the circadian rhythm treatment apparatus may contain software, a micro-controller, one or more LEDs, other light sources, some sensors for movement, physiological parameters, and/or sleep environment. There may be an audio module that delivers different audio sounds that played with (e.g., synchronized with) the circadian rhythm disorder treatment program (e.g., light program).

System 600 may be an embodiment of any of, or any combination of, systems 100, 200, 300, and/or 500. System 600 may be located in and/or used with systems 100, 200, 300, computing device 501 and/or a device in cloud 508.

Power 602 may include a battery, a USB port, an adaptor and/or an interface to an adaptor for converting one form of electricity (e.g., from a wall, 120 volts AC) to a form of electricity used by system 600 (e.g., 6 volts DC). In an embodiment, the power 602 may include a charger (e.g., a photocell) that charges from light, radio waves received, and/or other electromagnetic waves.

Near field communications 604 may receive and transmit communications via a near filed communications method. For example, near field communications 604 may use WiFi or UltraHigh Frequency (UHF) communications (e.g., using the Bluetooth standard or Bluetooth Low Energy). Near field communications 604 may be used for communicating with a wearable device (e.g., if system 600 is an embodiment of system 500) and/or may be in a wearable device or other device (e.g., if system 600 is an embodiment of system 100, 200, and/or 300), for example. Environment sensors 606 sense the environmental conditions, such as temperature and/or lighting of the environment. The environment sensor 606 may also sense if the wearable device is being worn by the user and use the data to inform and adjust the light program. Optionally, by detecting, environmental conditions (e.g., temperature, wind, noise, and/or movements), system 600 may determine whether disruptions in sleep are due to the environmental conditions (e.g., one-time disruptions in the environmental conditions) or whether the treatment window needs to be altered for a specific user and the environment in which the user ordinarily sleeps. For example, if even the environmental conditions are ordinarily not conducive for sleep and the user is not sleeping well, there may be no need to adjust the light program. By contrast if the environmental conditions for sleep are good and the user is not sleeping well, the treatment window may need to be altered. Sleep sensors 608a may include a collection of sensors used to determine the sleep/wake and/or the sleep stages and quality of the user's sleep. Sleep sensors 608a may include one or more accelerometers and gyroscope, one or more actigraphy, and/or one or more EEG sensors. Pulse sensor 608c may sense the user's pulse rate to determine the sleep/wake states. The pulse rate may monitored by detecting a pulsation in the temple area, eyes and/or wrist. Eye motion sensor 608d may detect the blinking of the eyes, rapid eye movement, and/or other eye movement to determine sleep/wake states and sleep stages. Actigraphy which monitors activities and lighting condition can be used to determine the circadian phase and the sleep/wake states. Body motion sensor 608e may detect movements of the head and/or movements of the rest of the body, so as to for example, thereby detect whether the user is tossing and turning more than usual during sleep. Breathing sensor 608f may determine whether the user is breathing while sleeping (if the user is not breathing the user may not feel rested despite the treatment) and/or the rate at which the user is breathing (as an indicator of sleep/wake state and sleep stages). Breathing sensor 608f may include a microphone for detecting the breathing pattern. Microphone 608g may detect noises associated with the environment, body movements (e.g., is the user tossing and turning) and/or breathing (e.g., is the user snoring, breathing deeply, breathing rapidly, the length of time with no breathing). Microphone 608g may also detect vocal noises during sleep as some types of vocal noises may be indications of stress. Microphone 608g may be part of breathing sensor 608f.

Brainwave detector 608h may detect brainwave, which may be analyzed to modify the treatment regimen. Brainwave detector 608h may include several EEG sensors to determine sleep/wake state and sleep stages.

Light system 610 may deliver treatment, via delivering flashes and/or other light to the user's eyes. Left light 612 may deliver light treatment to the left eye, and right light 614 may deliver treatment to the right eye. Left light 612 may include a light source, lens, and a light pipe for directing the light from the light source to the left eye. Right light 612 may also include a light source, lens, and a light pipe for directing the light from the light source to the right eye. In an embodiment, each of left light 612 and right light 614 include their own light source (e.g., one or more LEDs or other lights). In another embodiment, light system 610 may include a central light source (e.g. one or more LEDs or other lights shared by both left light 612 and right light 614), and left light 612 and right light 614 may include optical fibers and/or light pipes to transmit the light form the light source to the right and left eyes. The central light source may be in addition to, or instead of, the light source of the left light 612 and/or right light 614.

Microprocessor 616a may run application 502, compute a treatment regimen, based on user input, and/or location. Microprocessor 616a may control power 602 (e.g., by regulating power), near field communications 604, environment sensors 606, sleep sensors 608a, temperature sensor 608b, pulse sensor 608c, eye motion sensor 608d, body motion sensor 608e, breathing sensor 608f, microphone 608g, pulse modulator 616b, treatment logic 616c, brightness control 616d, sound system 618, optional microphone 620, speaker 622, other communications 624, and/or other components 626.

Pulse modulator 616b modulates electrical pulses, which in-turn modulate the flashes of the light (e.g. light flashes) produced by light system 610 based on application 502, for example, and input from the user. Treatment logic 616c, is the logic that determines the brightness/intensity of the light, the frequency that lights are turned on and off (the frequency of the flashes, the duration of the flashes, and width of light trains, and/or the start time and stop time of the treatment, for example). Brightness control 616d controls the brightness of left light 612 and right light 614. Brightness control 614d may control the brightness/intensity of the lights based on the input of user in application 502. Sound system 618 may be used to receive voice commands, play music, and/or play messages (e.g., generated by application 502). Optional microphone 620 (if present) may be the same as, or different than, microphone 608g. Optional microphone 620 may be used for receiving commands and/or other input. Speaker 622 may be used for conveying information provided by application 502. Other communications 624 may include an interface to a network and/or a telephone signal receiver and transmitter. Other components 626 may include a key pad, a baseband processor, answering machine logic, and/or other applications, for example. Calendar 628 may be used to schedule circadian rhythm treatments. Contacts 630 may list contacts and the locations of the contacts. In an embodiment, the use may be able to choose a circadian rhythm treatment by scheduling a trip to a contact or personally significant location. For example, the user may indicate that the user will be visiting one of the contacts 630 (or another location previously visited) and a treatment regimen may be setup based on the location of the contact and/or a previous visit to the contact to a previous location. In other words, after a visit to a location and or a person, a schedule may be stored and reused in a subsequent trip by just selecting the person or contact visited. Global Positioning System 632 may schedule and/or adjust a circadian rhythm treatment based on the user's current location.

FIG. 7 shows a block diagram of a device 700 used in the system of FIGS. 1-6. The device 700 may include output system 702, input system 704, memory system 706, processor system 708, baseband processor system 712, input/output device 714, and/or database 716.

Device 700 is an example of a device that may be used for any of the system of FIGS. 1-6. Device 700 may be a mobile device (e.g., any of systems 100-600), tablet computer, laptop computer, a server, a device located in the cloud, and/or desktop computer.

Output system 702 may include any one of, some of, any combination of, or all of a monitor system, a handheld display system, a printer system, a speaker system, a connection or interface system to a sound system, an interface system to peripheral devices and/or a connection and/or interface system to a computer system, intranet, and/or internet, for example. Output system 702 may include near field communications 604, light system 610, left light 612, right light 614, sound system 618, and/or speaker 622, for example.

Input system 704 may include any one of, some of, any combination of, or all of a keyboard system, a mouse system, a track ball system, a track pad system, buttons on a handheld system, a scanner system, a microphone system, a connection to a sound system, and/or a connection and/or interface system to a computer system, intranet, and/or internet (e.g., IrDA, USB), for example. Input system 704 may include near field communications 604, environment sensors 606, sleep sensors 608a, temperature sensor 608b, pulse sensor 608c, eye motion sensor 608d, body motion sensor 608e, breathing sensor 608f, brain wave detector 608h, and/or optional microphone 608g.

Memory system 706 may include, for example, any one of, some of, any combination of, or all of a long-term storage system, such as a hard drive; a short-term storage system, such as random access memory; a removable storage system, such as a floppy drive or a removable drive; and/or flash memory. Memory system 706 may include one or more machine-readable mediums that may store a variety of different types of information. The term machine-readable medium is used to refer to any medium capable carrying information that is readable by a machine. One example of a machine-readable medium is a computer-readable medium. Another example of a machine-readable medium is paper having holes that are detected that trigger different mechanical, electrical, and/or logic responses. The term machine-readable medium also includes mediums that carry information while the information that is in transit from one location to another, such as copper wire and/or optical fiber. Memory system 706 may store machine instructions for implementing the application, via which the user enters information related to their sleep schedule and interacts with the hardware in the mask or glasses or other circadian rhythm therapy device. Memory system 706 may include logic (e.g., drivers and/or other logic) for controlling power 602, near field communications 604, environment sensors 606, sleep sensors 608a, temperature sensor 608b, pulse sensor 608c, eye motion sensor 608d, body motion sensor 608e, breathing sensor 608f, microphone 608g, brain wave detector 608h, light system 610, left light 612, right light 614, sound system 618, optional microphone 620, and/or speaker 622. Memory system 706 may include logic (e.g., drivers and/or other logic) for implementing pulse modulator 616b, treatment logic 616c, and/or brightness control 616d.

Processor system 708 may include any one of, some of, any combination of, or all of multiple parallel processors, a single processor, a system of processors having one or more central processors and/or one or more specialized processors dedicated to specific tasks. Also, processor system 708 may include one or more Digital Signal Processors (DSPs) in addition to or in place of one or more Central Processing Units (CPUs) and/or may have one or more digital signal processing programs that run on one or more CPU. Processor system 708 may compute the circadian rhythm disorder treatment program and may run the application gathers the information about the circadian rhythm of the user and that computes the circadian rhythm disorder treatment program. Processor system 708 may include microprocessor 616a.

Baseband processor system 712 is optional and baseband processor system 712 controls functions related to the telephony. Baseband processor system 712 may include one or more processors. For example, baseband processor system 712 processes incoming phone signals converting the signals into voice messages and/or ring tones and converting user entered key pad sequences into signals representing the dialing a phone number and user entered voice/audio messages, via a microphone, as part of a phone conversation. Since telephone conversations and telephone messaging works better when processed in a fast speed, the baseband processor system is a processor system that is dedicated to the telephone functions. Baseband processor system 712 may function independently of processor system 708 and/or may have a master slave relationship with processor system 708, in which baseband processor system 712 is subservient to processor system 708. In an embodiment, baseband processor system 712 is subservient to processor system 708 for some functions, whereas processor system 708 is subservient to baseband processor system 712 for other functions.

Input/output system 714 may include devices that have the dual function as input and output devices. For example, input/output system 714 may include one or more touch sensitive screens, which display an image and therefore are an output device and accept input when the screens are pressed by a finger or stylus, for example. The touch sensitive screens may be sensitive to heat and/or pressure. One or more of the input/output devices may be sensitive to a voltage or current produced by a stylus, for example. Input/output system 714 is optional, and may be used in addition to or in place of output system 702 and/or input device 704. Input/output system 714 may include near field communications 604, environment sensors 606, sleep sensors 608a, temperature sensor 608b, pulse sensor 608c, eye motion sensor 608d, body motion sensor 608e, breathing sensor 608f, microphone 608g, brain wave detector 608h, light system 610, left light 612, right light 614, brightness control 616d, sound system 618, optional microphone 620 and speaker 622, other communications 624, and/or other components 626.

Database 716 may be used to store schedules for multiple users. For example, system 700 may be a device connected to the cloud and multiple user may enter their sleep schedules and any of devices 100-300 or 500 may connect, via cloud 508, to system 700, causing any of the systems 100-300 or 500 to produce the appropriate flashes to adjust a circadian rhythm. Database 716 may be located in the cloud remotely from the rest of system 700 or with system 700, which itself may be located in the cloud.

Processor system 708 and baseband processor system 712 are communicatively linked to one another and to output system 702, input system 704, memory system 706, and/or input/output system 714. In formation may be passed, via processor system 708, from any of output system 702, input system 704, memory system 706, and/or input/output system 714 to another of output system 702, input system 704, memory system 706, and/or input/output system 714. Processor system 708 and baseband processor system 712 are may be linked to output system 702, input system 704, memory system 706, and/or input/output system 714 by any one of, some of, any combination of, or all of electrical cables, fiber optic cables, and/or means of sending signals through air or water (e.g. wireless communications), or the like. Some examples of means of sending signals through air and/or water include systems for transmitting electromagnetic waves such as infrared and/or radio waves and/or systems for sending sound waves. In other embodiments, information may be shared between output system 702, input system 704, memory system 706, and/or input/output system 714 without participation of the processor system.

Database 716 may store information about users, contacts, and/or sleep schedules. In an embodiment in which system 700 is a device in the cloud (e.g. cloud 508) or a system that includes many devices, database 716 may store information related to multiple users, in which each user to limited to which part of the database 716 that user can access. In an embodiment in which system 700 is (additionally or alternatively) part of systems 100, 200, 300, and/or 400, database may be store information about one user or of multiple that users that shares the system.

FIG. 8 shows an example of a screenshot of an embodiment of page 800 for communicatively connecting the system of FIGS. 1, 2 and/or 3 to the system of FIG. 5, prior to the communication being established. In the embodiment of screenshot 800, the system of FIGS. 1, 2 and/or 3 is a mask, and that mask and the system 500 are not currently connected (or “paired”). Page 800 indicates that the system of FIGS. 1, 2, and/or 3 has been detected and provides a link, which when selected puts system 500 in communication with the system of FIGS. 1, 2, and/or 3. Page 800 may include a link or a field 802, which when selected connects system 500 to system 100, 200, or 300. Alternatively, in an embodiment, the user may be able to tap or otherwise select any portion of the screen and/or any key of a keypad associated with page 800 and/or system 500.

FIG. 9 shows an example of a screenshot of an embodiment of page 900 for communicatively connecting the system of FIGS. 1, 2 and/or 3 to the system of FIG. 5, after the communication was established. Page 900 is essentially the same as page 800, except in page 900 the user has already tapped (or otherwise selected the link that causes the communicative connection between the system of FIGS. 1, 2, and/or 3 and system 500). Whereas, page 900 indicates (e.g., via message 902) that the connection has been made and the system 100, 200, and/or 300 are “paired” with system 500.

FIG. 10 shows an example of a screenshot 1000 of a page for entering personal information for personalizing the application (e.g., as part of step 402, 404 or 406). In the example of screenshot 1000, the information requested includes birthdate in field 1002, gender in field 1004, eye color in field 1006, typical bedtime 1008, typical wake time in field 1010, and/or light sensitivity 1012. The information provided, via the page of screenshot 1000, may be used in monitoring the sleep of a user and determining whether a change in the sleep therapy needs to be made. Since heart rates, breathing patterns, and brain waves may differ according to age and gender, the birthdate and gender may be used to determine whether the user is in a deep sleep, and/or whether the user's sleep has been disturbed (e.g., as a result of the intensity of the flash). The user's typical bedtime and wake up time may be used to determine when to apply light flashes to adjust the user's wake time, causing the user to wake earlier or later, depending on whether the user needs to wake up earlier or later as a result of user's current work shift or travel schedule.

FIG. 11 shows an example of a screenshot of a page 1100 for displaying information for identifying the hardware and software of the system of FIG. 1, 2 or 3. The information displayed may include a device name (in field 1102), a serial number (in field 1104) of system 100, 200 and/or 300, an identifier (in field 1106) of the system of user 100, 200, an identifier (in field 1108) of the firmware running in system 100, 200, and/or 300, and/or 300, an identifier (in field 1110) of the software running on system 500 and/or 300, and/or an identifier (in field 1112) of the hardware running on systems 100, 200, 300, 400, 500, 600, and/or 700 (which may identify the version of systems 100, 200, 300, 400, 500, 600, and/or 700 currently in use). In the example of FIG. 11, page 1100 also includes an indicator (e.g., indicator 1114) for the level of the battery that powers system 100, 200, and/or 300 and/or a link (e.g., link 1116) for testing the lights used for producing the flashes (which may be LEDs). In an embodiment, indicator 1114 indicates the status of the battery (e.g., that the battery is not connected, is at 100%, or is weak) based on a communication from system 100, 200, and/or 300 to system 500 regarding the status of the battery. In the example of FIG. 11, page 1100 may include a link 1118 for communicatively connecting (e.g., “pairing”) system 100, 200, and/or 300 with system 500.

FIG. 12 shows an example of a screenshot 1200 of a page for choosing whether to view or edit the profile information of FIGS. 10 and/or 11 (e.g., via the edit my mask link 1202) or to review sleep schedules and light programs that have been entered, such as via the my programs link (1204). Screenshot 1200 may also have links for connecting to a help center (1208), accessing a tutorial (1206), and/or to chat (1210) with someone regarding how to use the system. Selecting my profile link 1201 may bring the user to a page for reviewing the user's profile.

Page 1200 may include Home link 1216, Schedule link 1218, Profile/setting-link 1220, Status link 1222.

Home link 1216, when selected may bring the user to the home page of the application, where the user may follow the instructions of the program to adjust the user's circadian rhythm. Schedule link 1218, when selected, may allow the user to input travel or shift schedules in order to calculate the circadian rhythm adjusting program. In an embodiment, the user may input the travel schedules including departure and arrival locations, dates, and times. In an embodiment, the user may indicate the night shifts on the calendar. In an embodiment, user may select dates and times to indicate the desired sleep schedules (e.g., when the user wishes to go to sleep and when the user wishes to wake up). Profile/settings link 1220, when selected may allow the user to set up personal profile, review past and/or upcoming schedules, and get help on how to use the mask. Status link 1222, may display information and status about the software and the hardware.

FIG. 13 is a screenshot of an example of Schedule page 1300. In an embodiment of the user interface of the Schedule page 1300, Schedule page 1300 may have a menu for choosing a type of circadian rhythm treatment. The page may include a trip option (e.g., schedule trip link 1302), shift sleep option (e.g., shift sleep link 1304) for changing sleep schedules, night shift option (e.g., night shift scheduling link 1306) for adjusting circadian rhythm for multiple night shifts. The trip option (e.g., schedule trip link 1302) may shift the user circadian rhythm based on a scheduled trip. The sleep shift (e.g., shift sleep link 1304) option may shift the sleep schedule of a user earlier or later without traveling. The night shift option (e.g., schedule night shift link 1306) may suggest a user's sleep schedule and a circadian adjusting program based on night shifts. There also may be options for choosing sleep therapies designed for specific sleep ailments, such as to accommodate the Daylight saving schedules or to alleviate seasonal affected disorders (SAD). The specific names of the options on the menu may be different from what are shown in the screenshot example.

FIG. 14 shows an example of a screenshot of a page 1400 for choosing the locations that the user plans to visit. In an embodiment, the user may select where the user plans to visit by selecting the airport (field 1402) nearest to the location that the user plans on visiting, by keypad 1404 and/or by selecting an the airport (or other location from a list of locations). In other embodiments, the user may select other indications of the location the that user plans on visiting, such as by entering the name of a city and country, by entering a flight number, by entering a street address, by entering a longitude and latitude of the location to be visited, and/or by entering global positioning coordinates of the location to be visited.

FIG. 15 shows an example of a screenshot of a page 1500 for choosing departure date (via field 1502) and departure time (via field 1504) for a particular location that the user intends to visit.

FIG. 16 shows an example of a screenshot of a page 1600 for choosing arrival date (via field 1602) and arrival time (via field 1604) and date for a particular location that the user intends to visit. The user also has the option of entering a return trip or a multi-leg trip via selection field 1606.

FIG. 17 shows an example of a screenshot of a page 1700 showing a circadian rhythm adjusting program for reducing jet lag after the trip has been entered. In an embodiment, there may be a page for adjusting circadian rhythm based on a scheduled trip. The page 1700 for adjusting the circadian rhythm may include an indicator 1702 that indicates the number of days over which the change is scheduled to occur. Page 1700 may include an indicator for a location 1704, day indicators 1706a and 1706b, and bedtime indicator 1708a-b and wake time indicator 1710a-b. The location indicator 1704 may show the location to which the user has chosen to travel and/or the location from which the user is traveling. The day indicators 1706a and 1706b explain which nights the user should use the device. The bedtime and wake time indicators may indicate the times at which the user is supposed to or planning to go to sleep and wake up on the each day of the trip. Since in this example of the user is planning to shift their circadian rhythm over a two-day period, the wake times and bedtimes may not be the times that the user plans to wake up and go to sleep during the rest of the trip. The user can adjust their bedtime and wake time on each individual day based on their updated schedule or activities. In one embodiment, the suggested bedtime and wake times can be adjusted based on user feedback or data collected from the sensors. In one embodiment, there may not be a page with information of the circadian rhythm adjusting program, and the system delivers the light treatment automatically and dynamically.

There may be an embodiment of a page for adjusting circadian rhythm based on a scheduled trip, which may include travel-to-location link, a related service link, and/or help link (in addition to the other links common to multiple pages). Travel-to-location link, when selected provides one or more fields, via which the user may enter a location that the user plans on traveling to and/or the user may enter the location from which the user is traveling from. The related service link, when selected, links the user to a page for requesting a related service. For example, when planning a trip, the user may select the related service link to reserve a rental car or buy an airline ticket. The help link, when selected, may bring the user to a page for receiving help about how to use the application.

One typical circadian rhythm misalignment is caused by night shifts. FIG. 18 shows a screenshot of an example of a page 1800 of a calendar for entering work shift schedules that occur at various times, prior to scheduling the shifts. On age 1800 links 1804a-c are shown for adding shifts. For example, the links 1804a-c may be plus sign inside circles (in other embodiments other icons maybe used instead of the plus signs). For example, the links 1804a-c may be plus sign inside circles to indicate where to add shifts. Once the plus signs are tapped upon, a block of shift shows up on the date accordingly (FIG. 19), indicating the times of shifts with the start of the shift, the duration of the shift, and the end of the shift. In an embodiment, it is possible to enter two or more, optionally side-by-side, sleep schedules for the user to choose between, which may assist the user in planning sleep schedules and/or allow multiple user to use the same system. Page 1800 includes list 1802, which is a list of dates and times at which shifts have been scheduled, e.g. in the form of a calendar. In an embodiment, there may not be a page for shift schedule input. The shift schedules may be automatically captured from work or personal calendar applications.

FIG. 19 shows a screenshot of an example of a page 1900 shows an example of a calendar for entering work shift schedule that occur at various times, after some shifts have been scheduled. FIG. 19 shows work schedule information related to multiple shifts that occur at various times. The page 1900 may be the same as page 1800 except that the shifts were scheduled on page 1900. Page 1900 indicates the times of shifts blocked off with blocks 1904a-c, which indicate the start of the shift, the duration of the shift, and the end of the shift. Although page 1900 has only three shifts scheduled, any number of shifts may be shown. The user may be able to drag the start and the stop of 1904a-c on the calendar to adjust the shift start and stop time.

FIG. 20 shows a screenshot of an example of a page 2000, having a field 2002 for entering a desired number of hours the user would like to sleep between the shifts.

FIG. 21A shows a screenshot 2100 of an example of a page showing a circadian rhythm adjusting program for the night shifts after the schedule has been entered. FIG. 21B shows a portion of the schedules in FIG. 21A. The screenshot 2100 includes an indication of the number the duration of time over which the multi-shift period occurred 2102, the number of shifts included in the schedule 2104, and the schedule for each sleep period, which in the example of FIG. 21B includes sleep periods 2106a-d. In one embodiment, there may not be a page with information of the circadian rhythm adjusting program, and the system delivers the light treatment automatically and dynamically.

The user may also use the system to change sleep schedules without traveling or working on night shifts. FIG. 22 shows a screenshot of an example of a page 2200, having a field 2202 for entering the user's current wake time.

FIG. 23 shows a screenshot of an example of a page 2300, having a field 2302 for entering the user's desired wake time.

FIG. 24 shows a screenshot of an example of a page 2400, having a field 2402 for entering when the user wants to start the new sleep schedule.

FIG. 25 shows a screenshot 2500 of an example of a page 2500 showing a circadian rhythm adjusting program for changing the sleep schedule after the desired schedule has been entered. The screenshot 2500 includes an indication of the schedule change 2502, the number the duration of time over which the adjustment occurs 2504, and the schedule for each sleep period, which in the example of FIG. 25 includes sleep periods 2506. In one embodiment, there may not be a page with information of the circadian rhythm adjusting program, and the system delivers the light treatment automatically and dynamically.

The inventor has tested the efficacy of one form of such circadian adjustment system described in FIGS. 1-11 on volunteer testers, using the application to reduce jet lag. FIG. 26 shows a bar chart 2600 comparing the number of days to get over from jet lag based on the number of time zones traversed, with and without the system of FIGS. 1-11. Bar chart 2600 includes time axis 2602 and time zone axis 2604. Time axis 2602 indicates the time-period during which the user felt discomfort as a result of their circadian rhythm being less than ideal, and the time zone axis 2604 indicates the number of time zones that were traveled. For each group of times zones, there are two bars one representing the number of days of discomfort with the system and one representing the number of days of discomfort without using the system. The user of the system experienced discomfort in around 50% shorter periods than those that did not use the system.

FIG. 27 shows a bar chart 2200 comparing the number of days experiencing jet lag, based on the direction traversed, with and without the system of FIGS. 1-11. Bar chart 2700 includes a time axis 2702 and an x-axis 2704. The jet lag axis indicates the number of days that the individual experiences jet lag. The x-axis 2704 labels two pairs of bars. One pair of bars are for outbound trips and one pair of bars are for inbound trips. Outbound trips are trips to a location that the individual is not usually located from a location where the user lives, and inbound trips are trips returning to a location where the user usually lives. No matter which direction, the user experiences jetlag in about 50% fewer days more when using the system.

FIG. 28 shows a bar chart 2800 of how users rated how much an embodiment of the system of FIGS. 1-11 helped reducing jet lag for their trips. Axis 2802 indicates the number of users choosing each rating score and axis 2804 shows the rating that the users assigned to the embodiment of the system, with 0 indicating “Not helpful at all” while 10 indicating “I have no jet lag”. Bar chart 2800 shows that vast majority gave the system a favorable rating. As can be seen, most user rated the system with an 8/10, many users rates the system with a 10/10, and the vast majority of users rated the system with 7 out of 10 or higher.

FIG. 29 shows a bar chart 2900 based on chart 2800 in FIG. 28, showing how many users assigned a rating that falls into one of the categories of positive, neutral, negative, and lights being too bright. Axis 2902 indicates how many users gave a given rating, and axis 2904 has the four labels of positive, neutral, negative, and lights being too bright. Positive includes ratings 6-10, Negative includes ratings 0-4, and Neutral includes ratings equal to 5, with 0 indicating “Not helpful at all” while 10 indicating “I have no jet lag”. As can be seen the vast majority of user gave the system 100 a positive rating.

FIG. 30 shows a bar chart 3000 of whether users would recommend an embodiment of the system of FIGS. 1-11, with 0 indicating “Not at all likely” while 10 indicating “Extremely likely.” Axis 3002 indicates the number of user that gave a particular rating, and axis 3004 shows the rating given by the users. Bar chart 3000 shows that the vast majority of users would recommend the system to a friend.

FIG. 31 shows how users rated the comfort of an embodiment of the system of FIG. 1-11, with 0 indicating “Very uncomfortable” while 10 indicating “Very comfortable”. Axis 3102 indicates the number of user that gave a particular rating, and axis 3104 of shows the rating given by the users. FIG. 31 shows that the overall majority of users found the mask comfortable.

### ALTERNATIVES AND EXTENSIONS

Each embodiment disclosed herein may be used or otherwise combined with any of the other embodiments disclosed. Any element of any embodiment may be used in any embodiment.

Although the invention has been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention.

1. A system comprising:
a processor system comprising one or more processors;
a memory system that stores one or more machine instructions for implementing a method including at least
detecting, via one or more detectors information related to a circadian rhythm of a user;
altering, by the processor system, instructions for producing a pattern for activating a light, based on the information about the circadian rhythm that was detected, therein altering the pattern for activating the light to adjust circadian rhythms.

2. The system of claim 1, the pattern including light pulses.

3. The system of claim 1, wherein the pattern includes at least flashes of light that have a luminous flux of 50 lux to 4000 lux when measured from 0 to 5 mm from a user's eye.

4. The system of claim 1, wherein the pattern includes at least flashes of light having a frequency of between 1 flash per 9 seconds and 1 flash per 120 seconds.

5. The system of claim 1, wherein the pattern includes a train of flashes that lasts 4 hours or less.

6. The system of claim 1, wherein the pattern includes flashes having a full spectrum of wavelengths between about 380 to 750 nm.

7. The system of claim 1, wherein the pattern includes flashes of a wavelength within between about 380 nm to 750 nm.

8. The system of any of claims 1, wherein the pattern includes flashes of white light.

9. The system of claim 1, wherein the pattern includes flashes of light, having a luminous flux of at least 50 lux, when measured at a surface of an open eye of a user.

10. The system of claim 1, wherein the pattern includes flashes having a luminous flux of at least 500 lux measured at an eyelid of a closed eye.

11. The system of any of claims 1, wherein the pattern includes pulses that are repeated at least once per 120 seconds for at least a subset of a treatment.

12. The system of any of claims 1, wherein, the pattern includes the pulses that are projected over a time period between about 10 and 240 minutes.

13. The system of any of claims 1, wherein the pattern includes pulses that are projected during CT15-CT24 and CT24-CT8, in which CT0 is defined as the cross-over point of the human Phase-response curve or as the time of the fitted minimum of unmasked core body temperature.

14. The system of claim 1, wherein the pattern includes a part of a circadian rhythm treatment of a user that is scheduled to occur while the user is scheduled to be sleeping.

15. The system of claim 1, further comprising a sensor that senses whether or not the circadian treatment apparatus is being worn by the user, and treatment logic that modifies a treatment based on this information.

16. The system of claim 1, further comprising a sensor that detects eye movement.

17. The system of claim 1, further comprising a sensor that senses body movement.

18. The system of claim 1, further comprising a microphone and treatment logic that modifies a treatment based on breathing patterns.

19. The system of claim 1, further comprising a microphone and treatment logic that modifies a treatment based on environmental noises.

20. The system of claim 1, further comprising a light detector and treatment logic that modifies a treatment based on light exposure.

21. The system of claim 1, further comprising a motion detector and treatment logic that modifies a treatment based on motions of the body detected.

22. The system of claim 1, further comprising a brainwave detector and treatment logic that modifies a treatment based on brainwave patterns.

23. The system of claim 1, further comprising a heart rate detector and treatment logic that modifies a treatment based on the heart rate detected.

24. The system of claim 1, further comprising an eye movement detector and treatment logic that modifies a treatment based on eye movements detected.

25. The system of claim 1, the method further including computing a circadian rhythm based on the information related to the circadian rhythm; the altering being based on the information related to the circadian rhythm by being based on the circadian rhythm computed.

26. The system of claim 1, the altering of the pattern including altering a frequency of the flashes.

27. The system of claim 1, the altering of the pattern including altering an intensity of the flashes.

28. The system of claim 1, the altering of the pattern including altering an duration of the flashes.

29. The system of claim 1, the altering of the pattern including altering a timing of the flashes.

30. The system of claim 1, providing information on whether a treatment program is properly applied.

31. A system comprising:
a processor system comprising one or more processors;
a memory system that stores one or more machine instructions for implementing a method including at least detecting, via one or more detectors, information related to sleep of a user;
altering, by the processor system, instructions for producing a pattern for activating a light, based on the information about the sleep detected, therein altering the pattern for activating the light to adjust circadian rhythms.

32. A system comprising:
a processor system including one or more processors;
a memory system that stores a nonvolatile memory one or more machine instructions, which when invoked by the system causes the system to implement a method including at least
presenting, on a display, an interface for entering information related to a circadian rhythm;
receiving information, via an input module, related to the circadian rhythm;
adjusting parameters, by the processor system, of machine instructions for producing light flashes for activating a light to adjust circadian rhythm.

33. A method comprising:
projecting light pulses, the light pluses having a flash duration of 1 microseconds to 500 milliseconds to adjust a circadian rhythm of one exposed to the pulses.
